SEQUENCE LISTING (110 sequences)

SEQ ID NO: 1 - protein, 4 aa, Artificial Sequence
  synthetic protein
MKTK

SEQ ID NO: 2 - DNA, 18 bp, Artificial Sequence
  synthetic DNA
gctagcatgaagaccaag

SEQ ID NO: 3 - protein, 5 aa, Artificial Sequence
  synthetic protein
MGKTK

SEQ ID NO: 4 - DNA, 20 bp, Artificial Sequence
  synthetic DNA
gctagcatgggaaagacagg

SEQ ID NO: 5 - DNA, 51 bp, Artificial Sequence
  synthetic DNA
tacagtgccttgcgaaagtattcggcccccttgaacttttcgaccttttgc

SEQ ID NO: 6 - DNA, 51 bp, Artificial Sequence
  synthetic DNA
tacagtgccttgcgaaagtattcggcccccttgaactttgcgaccttttgc

SEQ ID NO: 7 - DNA, 51 bp, Artificial Sequence
  synthetic DNA
tacagtgccttgcgaaagtattcggcccccttgaacttttcaaccttttgc

SEQ ID NO: 8 - DNA, 51 bp, Artificial Sequence
  synthetic DNA
tacagtgccttgcaaaagtattcggcccccttgaacttttccacattttgc

SEQ ID NO: 9 - DNA, 50 bp, Artificial Sequence
  synthetic DNA
tacagtgccttgcragagtattcatcccccttgcgtttttcctattttgt

SEQ ID NO: 10 - DNA, 51 bp, Artificial Sequence
  synthetic DNA
tacagtgccttgcataagtattcaccccccttgccgtttttcctattttgt

SEQ ID NO: 11 - DNA, 51 bp, Artificial Sequence
  synthetic DNA
tacagtgccttgcataagtattcaccccccttggctttttacctattttgt

SEQ ID NO: 12 - DNA, 51 bp, Artificial Sequence
  synthetic DNA
tacagtgccttgcataagtattcaccccccttggacttttctacattttgt

SEQ ID NO: 13 - DNA, 48 bp, Artificial Sequence
  synthetic DNA
aaaactgaaaaattgggcgtgcaaaattattcagcccctttactttca

SEQ ID NO: 14 - DNA, 48 bp, Artificial Sequence; identical to SEQ ID NO: 13
  synthetic DNA

SEQ ID NO: 15 - DNA, 49 bp, Artificial Sequence
  synthetic DNA
aaaaactgaaatattgggcgtgcaaaattattcagcccctttactttca

SEQ ID NO: 16 - DNA, 49 bp, Artificial Sequence
  synthetic DNA
ataactgcaaagtggggtgtgcgtaattattcagccccctttggtctga

SEQ ID NO: 17 - DNA, 39 bp, Artificial Sequence
  synthetic DNA
aagtggtgcgtgcatatgtattcaccccctttgctatga

SEQ ID NO: 18 - DNA, 50 bp, Artificial Sequence
  synthetic DNA
aaactmcagaaaagtggtgcgtgcatatgtattcaccccctttgctatga

SEQ ID NO: 19 - DNA, 47 bp, Artificial Sequence
  synthetic DNA
aaaactgataattggcatgtgcgtatgtattcacccctttgttatga

SEQ ID NO: 20 - DNA, 48 bp, Artificial Sequence
  synthetic DNA
aaatctgaaaagtgttgagtgcatatgtattcaccccctttactgtga

SEQ ID NO: 21 - protein, 60 aa, Artificial Sequence
  synthetic protein
MKNKEHTRQVRDTVVEKFKAGFGYKKISQALNIPRSTVQAIILKWKEYQTTANLQRPGRP

SEQ ID NO: 22 - protein, 60 aa, Artificial Sequence
  synthetic protein
MKNKBHIRQVRDTVVKKPKAGFGYKKISQALNIPRSTVQAIILKWKEYQTTANLPRPGRP

SEQ ID NO: 23 - protein, 60 aa, Artificial Sequence
  synthetic protein
MKNKEHTRQVRDTVVEKFKXGFGYKKISQALNIPRSTVQAIILKWKEYQTTANLQRPGRP

SEQ ID NO: 24 - protein, 60 aa, Artificial Sequence
  synthetic protein
MKSKBHTRQVRDKVIEKPKAGLGYKKISKALNIPRSTVQAIIQKWKEYGTTVNLPRQGRP

SEQ ID NO: 25 - protein, 59 aa, Artificial Sequence
  synthetic protein
MKTKELSKQVRDKVVEKYRSGLGYKKISETLNIPQSTIKSIIKWKEYGTTTNLPKEGRP

SEQ ID NO: 26 - protein, 60 aa, Artificial Sequence
  synthetic protein
MKTKELSKQVRDKVVEKYRSGLGYKKISETLNIPRSTIKSIIKKLKEYGTTTNLPREGRP

SEQ ID NO: 27 - protein, 61 aa, Artificial Sequence
  synthetic protein
MKTKELSKQVRDNVVEKYKSGLGYKKISKSLMIPRSTIKSIITKWEKEHGTTANLPRDGRT

SEQ ID NO: 28 - protein, 60 aa, Artificial Sequence
  synthetic protein
MKTKELTKQVRDKVVEKYEAGLGYKKISRALNISLSTIKSIIRKWKEYGTTANLPRGGRP

SEQ ID NO: 29 - protein, 339 aa, Artificial Sequence
  synthetic protein
MKTKELTKQVRDKVVEKYEAGLGYKKISRALNISLSTIKSIIRKWKEYGTTANLPRGGRPPKLKSRTRRKLIREATRRPMVTLEELQRSTAEVGESVHRTTISRLLHKSGLYGRVARRKPLLKGIHKKSRLEFARSHVGDTANMWKKVLWSDETKIELFGLNAKRYVWRKPNTAHHPEHTIPTVKHGGGSIMLWGCFSSAGTGKLVRIEGKMDGAKYREILEENLMQSAKDLRLGRRFIFQQDNDPKHTARATKEWFGLKNVNVLKWPSQSPDLNPIENLWQDLKIAVHRRSPSNLTELHLFCQEEWTNLSISRCAKLVETYPKRLAAVIAAKGGSTKY

SEQ ID NO: 30 - protein, 340 aa, Artificial Sequence
  synthetic protein
MGKSKEISQDLRKKIVDLHKSGSSLGAISKRLKVPRSSVQTIVRKYKHHGTTQPSYRSGRRRVLSPRDERTLVRKVQINPRTTAKDLVKMLEETGTKVSISTVKRVLYRHNLKGRSARKKPLLQNRHKKARLRFARAHGDKDRTFWRNVLWSDETKIELFGHNDHRYVWRKKGEACKPKNTIPTVKHGGGSIMLWGCGAACGTGALHKIDGIMRKENYVDILKQHLKTSVRKLKLGRKWVFQQDNDPKHTSKHVRKWLKDNKVKVLEWPSQSPDLNPIENLWAELKKRVRARRPTNLTQLHQLCQEEWAKIHPTYCGKLVEGYPKRLTQVKQFKGNATKY

SEQ ID NO: 31 - protein, 340 aa, Artificial Sequence
  synthetic protein
MPRPKEIQEQLRKKVIEIYQSGKGYKAISKALGIQRTTVRAIIHKWRRHGTVVNLPRSGRPPKITPRAQRRLIQEVTKDPTTTSKELQASLASVKVSVHASTIRKRLGKNGLHGRVPRRKPLLSKKNIKARLNFSTTHLDDPQDFWDNILWTDETKVELFGRCVSKYIWRRRNTAFHKKNIIPTVKYGGGSVMVWGCFAASGPGRLAVIKGTMNSAVYQEILKENVRPSVRVLKLKRTWVLQQDNDPKHTSKSTTEWLKKNKMKTLEWPSQSPDLNPIEMLWYDLKKAVHARKPSNVTELGQFCKDEWAKIPPGRCKSLIARYRKRLVAVVAAKGGPTSY

SEQ ID NO: 32 - protein, 339 aa, Artificial Sequence
  synthetic protein
MXKSKEISXQLRKKVVEIYXSGXGYKAISKALXIXRSTVKSIIRKWKXHGTTXNLPRSGRPPKLSPRXXRKLIREVTKXPXTTAKELQKSLAEVGXSVHXSTIKRLLHKXGLHGRVARRKPLLXXKHKKARLXFARSHLDDXXXFWKNVLWSDETKIELFGXNXXRYVWRKKNTAXHPKNTIPTVKHGGGSIMLWGCFAAAGTGKLXKIDGXMXXAXYXEILKENLKXSVRXLKLGKWVFQQDNDPKHTSKATKEWLKXNKVKVLEWPSQSPDLNPIENLWXDLKKAVHARKPSNLTELHQFCQEEWAKIXPSRCAKLVEXYPKRLXAVIAAKGGATKY

SEQ ID NO: 33 - DNA, 1625 bp, Artificial Sequence
  synthetic DNA
tgccttgcataagtattcaccccctttggacttttctacattttgtcatggtataaccacagattaaaatttatttcatcgtgagtttatgtaatggaccaacacaaaatagtgcatcatttggaagtggggggaaatattacatggatttcacaattatttacaaataaaaatctgaaaagtgttgagtgcatatgtattcaccccctttactgtgaaacccctcacaaagatctggtgcgaccaattgcattcacaagtcacatttgcaagtcacataattagtaaatagggtccacctgtctgcaatttaatctcagtataaatacacctgttctgtgacggactcagagtttgttggagatcattactgaacaaacagcatcatgaagaccaaggagctcaccaaacaggtcagggataaagttgtggagaaatatgaagcagggttaggttataaaaaaatatccagagctttgaacatctctctgagcaccataaaatccatcataagaaaatggaaagaatatggcacaaccgcaaacctaccaagaggaggccgtccacccaaactgaagagtcggacaaggagaaaattaatcagagaagcaaccaggaggcccatggttactctggaggagttgcagagatccacagctgaggtgggagaatctgtccacaggacaactattagtcgtctactccacaaatctggcctttatggaagagtggcaagaagaaagccattgttgaaaggtatccataaaaaatcccgtttggagtttgccagaagccatgtgggagacacagcaaacatgtggaagaaggtgctctggtcagatgagaccaaaattgaactttttggcctcaatgcaaaacgatatgtgtggcgaaaacccaacactgcccatcaccctgagcacaccatcccaacagtgaaacatggtggtggtagcatcatgctgtggggatgcttctcttcagcaggtacagggaaactggtcagaatagagggaaagatggatggagccaaatacagggaaatccttgaagaaaatctgatgcagtctgcaaaagacttgagactggggcggaggttcatcttccagcaggacaatgaccctaaacatacagccagagctacaaaggaatggtttggattaaagaatgttaatgtcttaaaatggcccagtcaaagcccagacctcaatccaatagagaatctatggcaagacttgaagattgcggttcacagacggtctccatccaatctgactgagcttcatcttttttgccaagaagaatggacaaacctttccatctctagatgtgcaaagctggtagagacataccccaaaagacttgcagctgtaattgcagcgaaagggggttctaccaagtattgacacaggggggtgaatacttatgcacccaacagatgtcaacttttttgttctcattattgtttgtgtcacaataaaatttattttgcacctccaaagtactatgcatgttttgttgatcaaacgggaaaaagtttatttaagtctatttgaattccagttagtaacagtacataatgggaaaaagtccaaggggggtgaatacttatgcaaggcactgta

SEQ ID NO: 34 - DNA, 385 bp, Artificial Sequence
  synthetic DNA
tacagtgccttgcataagtattcaccccctttggacttttctacattttgtcatgctataaccacagattaaaatttatttcatcgtgagtttatgtaatggaccaacacaaaatagtgcatcatttggaagtggggggaaatattacatggatttcacaattatttacaaataaaaatctgaaaagtgttgagtgcatatgtattcaccccctttactgtgaaacccctaacaaagatctggtgcgaccaattgcattcacaagtcacatttgcaagtcacataattagtaaatagggtccacctgtctgcaatttaatctcagtataaatacacctgttctgtgacggactcagagtttgttggagatcattactgaacaaac

SEQ ID NO: 35 - DNA, 237 bp, Artificial Sequence
  synthetic DNA
ggttctaccaagtattgacacaggggggtgaatacttatgcacccaacagatgtcaacttttttgttctcattattgtttgtgtcacaataaaatttattttgcacctccaaagtactatgcatgttttgttgatcaaacgggaaaaagtttatttaagtctatttgaattccagttagtaacagtacataatgggaaaaagtccaaggggggtgaatacttatgcaaggcactgta

SEQ ID NO: 36 - DNA, 216 bp, Artificial Sequence
  synthetic DNA
tacagtgccttgcataagtattcaccccctttggacttttctacattttgtcatgctataaccacagattaaaatttatttcatcgtgagtttatgtaatggaccaacacaaaatagtgcatcatttggaagtggggggaaatattacatggatttcacaattatttacaaataaaaatctgaaaagtgttgagtgcatatgtattcacccccttt

SEQ ID NO: 37 - DNA, 217 bp, Artificial Sequence
  synthetic DNA
caggggggtgaatacttatgcacccaacagatgtcaacttttttgttctcattattgtttgtgtcacaataaaatttattttgcacctccaaagtactatgcatgttttgttgatcaaacgggaaaaagtttatttaagtctatttgaattccagttagtaacagtacataatgggaaaaagtccaaggggggtgaatacttatgcaaggcactgta

SEQ ID NO: 38 - DNA, 23 bp, Artificial Sequence
  synthetic DNA
tctccctttagtgagggttaatt

SEQ ID NO: 39 - DNA, 23 bp, Artificial Sequence
  synthetic DNA
tctccctatagtgagtcgtatta

SEQ ID NO: 40 - DNA, 32 bp, Artificial Sequence
  synthetic DNA
aaggatccgattacagtgccttgcataagtat

SEQ ID NO: 41 - DNA, 34 bp, Artificial Sequence
  synthetic DNA
aaggtaccgattacagtgccttgcataagtattc

SEQ ID NO: 42 - DNA, 27 bp, Artificial Sequence
  synthetic DNA
ccagtttgttcagtaatgatctccaac

SEQ ID NO: 43 - DNA, 25 bp, Artificial Sequence
  synthetic DNA
ccaggttctaccaagtattgacaca

SEQ ID NO: 44 - DNA, 26 bp, Artificial Sequence
  synthetic DNA
ccatctttgttaggggtttcacagta

SEQ ID NO: 45 - DNA, 30 bp, Artificial Sequence
  synthetic DNA
aaagctagcatgaagaccaaggagctcacc

SEQ ID NO: 46 - DNA, 26 bp, Artificial Sequence
  synthetic DNA
aaggatcctcaatacttggtagaacc

SEQ ID NO: 47 - DNA, 33 bp, Artificial Sequence
  synthetic DNA
aaagctagcatgggaaagaccaaggagctcacc

SEQ ID NO: 48 - DNA, 52 bp, Artificial Sequence
  synthetic DNA
tctccctttagtgagggttaattgatatctaatacgactcactatagggaga

SEQ ID NO: 49 - DNA, 35 bp, Artificial Sequence
  synthetic DNA
cctccactacgactcactgaagggcaagcagtcct

SEQ ID NO: 50 - DNA, 10 bp, Artificial Sequence
  synthetic DNA
aacaaccatg

SEQ ID NO: 51 - DNA, 31 bp, Artificial Sequence
  synthetic DNA
gtgttggtccattacataaactcacgatgaa

SEQ ID NO: 52 - DNA, 28 bp, Artificial Sequence
  synthetic DNA
gggtgaatacttatgcacccaacagatg

SEQ ID NO: 53 - DNA, 29 bp, Artificial Sequence
  synthetic DNA
gcatgacaaaatgtagaaaagtccaaagg

SEQ ID NO: 54 - DNA, 28 bp, Artificial Sequence
  synthetic DNA
cagtacataatgggaaaaagtccaaggg

SEQ ID NO: 55 - DNA, 20 bp, Artificial Sequence
  synthetic DNA
atgatgcagctggatccgat

SEQ ID NO: 56 - DNA, 20 bp, Artificial Sequence
  synthetic DNA
atcggtaccatttaaatctg

SEQ ID NO: 57 - DNA, 20 bp, Artificial Sequence
  synthetic DNA
ctacccagactcatttgatt

SEQ ID NO: 58 - DNA, 20 bp, Artificial Sequence
  synthetic DNA
actgggaaagtctcttggta

SEQ ID NO: 59 - DNA, 20 bp, Artificial Sequence
  synthetic DNA
tttcaattcttttgaatgta

SEQ ID NO: 60 - DNA, 17 bp, Artificial Sequence
  synthetic DNA
cctagaatagttgctgg

SEQ ID NO: 61 - DNA, 22 bp, Artificial Sequence
  synthetic DNA
gttggggaacttaaccttgaac

SEQ ID NO: 62 - DNA, 20 bp, Artificial Sequence
  synthetic DNA
gtatagaaaggatgtccgaa

SEQ ID NO: 63 - DNA, 20 bp, Artificial Sequence
  synthetic DNA
gtccagaagtgagttcagat

SEQ ID NO: 64 - DNA, 17 bp, Artificial Sequence
  synthetic DNA
gatcattgttagcacct

SEQ ID NO: 65 - DNA, 20 bp, Artificial Sequence
  synthetic DNA
gtttttatttatcttgagta

SEQ ID NO: 66 - DNA, 20 bp, Artificial Sequence
  synthetic DNA
taccatgaattggcactgct

SEQ ID NO: 67 - DNA, 18 bp, Artificial Sequence
  synthetic DNA
gatggttgcattaacatt

SEQ ID NO: 68 - DNA, 20 bp, Artificial Sequence
  synthetic DNA
tgtcctaaattatgcacaat

SEQ ID NO: 69 - DNA, 20 bp, Artificial Sequence
  synthetic DNA
agacatagatgttacatata

SEQ ID NO: 70 - DNA, 20 bp, Artificial Sequence
  synthetic DNA
gatttagtgtattgtagata

SEQ ID NO: 71 - DNA, 21 bp, Artificial Sequence
  synthetic DNA
tacatggtagtttaaaaatta

SEQ ID NO: 72 - DNA, 20 bp, Artificial Sequence
  synthetic DNA
catcactttgtatatggagc

SEQ ID NO: 73 - DNA, 18 bp, Artificial Sequence
  synthetic DNA
catctttttattgttagg

SEQ ID NO: 74 - DNA, 20 bp, Artificial Sequence
  synthetic DNA
gtaagtgtatatttcaaacc

SEQ ID NO: 75 - DNA, 20 bp, Artificial Sequence
  synthetic DNA
gcagaggcctgtgtcaggtt

SEQ ID NO: 76 - DNA, 20 bp, Artificial Sequence
  synthetic DNA
aatgtgagctgcaggcagag

SEQ ID NO: 77 - DNA, 20 bp, Artificial Sequence
  synthetic DNA
tcaaagcaagaaagatttat

SEQ ID NO: 78 - DNA, 20 bp, Artificial Sequence
  synthetic DNA
gctcgagtctctgcaacaaa

SEQ ID NO: 79 - DNA, 20 bp, Artificial Sequence
  synthetic DNA
gagtggctaagtaggatatt

SEQ ID NO: 80 - DNA, 20 bp, Artificial Sequence
  synthetic DNA
ggttctcaaagctaatagag

SEQ ID NO: 81 - DNA, 20 bp, Artificial Sequence
  synthetic DNA
tgttgtcaagtttattgata

SEQ ID NO: 82 - DNA, 20 bp, Artificial Sequence
  synthetic DNA
catcctttaataatgctttt

SEQ ID NO: 83 - DNA, 19 bp, Artificial Sequence
  synthetic DNA
cgcacaagtcgatagtatt

SEQ ID NO: 84 - DNA, 20 bp, Artificial Sequence
  synthetic DNA
tgctaaagtctctctgaaat

SEQ ID NO: 85 - DNA, 20 bp, Artificial Sequence
  synthetic DNA
ctacgtatagatttgactgg

SEQ ID NO: 86 - DNA, 20 bp, Artificial Sequence
  synthetic DNA
tacaaccttcctggggcggc

SEQ ID NO: 87 - DNA, 20 bp, Artificial Sequence
  synthetic DNA
gatgctagagaatcaacttt

SEQ ID NO: 88 - DNA, 19 bp, Artificial Sequence
  synthetic DNA
attccaaacttggtacatt

SEQ ID NO: 89 - DNA, 20 bp, Artificial Sequence
  synthetic DNA
taatagtgatgagtggtatc

SEQ ID NO: 90 - DNA, 20 bp, Artificial Sequence
  synthetic DNA
tctccactcaagaaaaatgg

SEQ ID NO: 91 - DNA, 20 bp, Artificial Sequence
  synthetic DNA
gcatccccacagacacacct

SEQ ID NO: 92 - DNA, 20 bp, Artificial Sequence
  synthetic DNA
cctgttcagtgcaggcacct

SEQ ID NO: 93 - DNA, 20 bp, Artificial Sequence
  synthetic DNA
cagctctccctctgcctccc

SEQ ID NO: 94 - DNA, 20 bp, Artificial Sequence
  synthetic DNA
ttataagaacactgatgatt

SEQ ID NO: 95 - DNA, 20 bp, Artificial Sequence
  synthetic DNA
tctatcattaccccatggcc

SEQ ID NO: 96 - DNA, 20 bp, Artificial Sequence
  synthetic DNA
gatcatgaaactgagtctta

SEQ ID NO: 97 - DNA, 20 bp, Artificial Sequence
  synthetic DNA
agaggagagaagggagcttt

SEQ ID NO: 98 - DNA, 19 bp, Artificial Sequence
  synthetic DNA
atacagctttcggtcaaaa

SEQ ID NO: 99 - DNA, 20 bp, Artificial Sequence
  synthetic DNA
tccctataagctctaccatg

SEQ ID NO: 100 - DNA, 19 bp, Artificial Sequence
  synthetic DNA
cctacagtcctagggcaga

SEQ ID NO: 101 - DNA, 20 bp, Artificial Sequence
  synthetic DNA
gcaaaccaccatggcatatg

SEQ ID NO: 102 - DNA, 20 bp, Artificial Sequence
  synthetic DNA
tagctatgtaacaaacatgc

SEQ ID NO: 103 - DNA, 20 bp, Artificial Sequence
  synthetic DNA
ttggtttgaataactggttt

SEQ ID NO: 104 - DNA, 20 bp, Artificial Sequence
  synthetic DNA
gttcaatgtcaaccctgcaa

SEQ ID NO: 105 - DNA, 20 bp, Artificial Sequence
  synthetic DNA
cccagaaaccagccataatc

SEQ ID NO: 106 - DNA, 20 bp, Artificial Sequence
  synthetic DNA
ctcatttagcaaaaatcatg

SEQ ID NO: 107 - DNA, 20 bp, Artificial Sequence
  synthetic DNA
tagttatttatactaaggtg

SEQ ID NO: 108 - DNA, 20 bp, Artificial Sequence
  synthetic DNA
aatgattgctgtcccactca

SEQ ID NO: 109 - DNA, 20 bp, Artificial Sequence
  synthetic DNA
ttacacatatgatgccatgc

SEQ ID NO: 110 - DNA, 20 bp, Artificial Sequence
  synthetic DNA
tctttattgttctgtagctt

(19) United States
(12) Patent Application Publication
(10) Pub. No.: US 2010/0105140 A1
(43) Pub. Date: Apr. 29, 2010

(54) PLAICE DNA TRANSPOSON SYSTEM

(76) Inventors: Scott C. Fahrenkrug, Minneapolis, MN (US); Karl J. Clark, Rochester, MN (US); Daniel F. Carlson, Inver Grove Heights, MN (US); Michael J. Leaver, Stirling (GB)

Correspondence Address: DARDI & HERBERT, PLLC, 220 S. 6TH ST., SUITE 2000, U.S. BANK PLAZA, MINNEAPOLIS, MN, 55402, US

(21) Appl. No.: 12/504,286

(22) Filed: Jul. 16, 2009

Related U.S. Application Data
(60) Provisional application No. 61/081,324, filed on Jul. 16, 2008.

(51) Int. Cl. C12N 15/85; C12N 9/12; C07H 21/00; C12N 5/00

(52) U.S. Cl. 435/455; 435/194; 536/23.2; 435/325

(57) ABSTRACT

This document describes the Passport transposon system and methods of making and using the same.

### CROSS-REFERENCE TO RELATED APPLICATIONS

This patent application claims priority to U.S. Provisional Ser. No. 61/081,324 filed Jul. 16, 2008, which is hereby incorporated by reference herein.

### TECHNICAL FIELD

The technical field relates to DNA transposon systems, and more particularly to using such transposon systems for expressing genes, mapping genes, mutagenesis, and introducing DNA into a host chromosome.

### BACKGROUND

Mobilization of transposons is hypothesized to contribute to the evolution of host genomes by several mechanisms, including; imperfect repair after excision, insertional mutagenesis, qualitative and quantitative changes in the regulation of adjacent gene expression, and even in the creation of new genes (Girard and Freeling, Developmental Genetics 1999, 25(4):291-296; Lander et al. Nature 2001, 409(6822):860-921.]. Tc1/mariner elements are found in phylogenetically diverse species, including fungi, plants, ciliates and animals [Plasterk et al. Trends Genet. 1999, 15(8):326-332; Robertson J. Insect Physiology 1995, 41(2):99-105.]. This family of DNA transposons is comprised of a transposase gene flanked by terminal inverted repeats and is non-conservatively mobilized by a cut-and-paste mechanism. The Tel/mariner transposases belong to a large family of enzymes, including Tn7, Tn10, Mu transposases and retroviral and retrotransposon integrases, characterized by a DDE/D motif involved in polynucleotidyl transfer reactions [Plasterk et al., supra].

### SUMMARY

A transposon-transposase system, referred to herein as “Passport” has been discovered. The components of the system were isolated for the first time from the fish Pleuronectes plates.

Tc1/mariner elements can be active in the soma and the germline. Therefore, regulation of transpositional activity is required for host viability, and by extension, transposon persistence [Hartl et al. Trends Genet 1997, 13(5):197-201]. Evolutionary periods of transpositional activity are thus interspersed with periods of stochastic loss [Jacobson et al. Proc. Natl. Acad. Sci. USA 1986, 83(22):8684-8688] and “vertical inactivation” of transposons, wherein only defective versions are preserved, containing frame-shifts, deletions, and missense mutations. Nonetheless, representatives of this family of transposons have been demonstrated to be active in nematodes [Collins et al. Genetics 1989, 121(1):47-55; Moerman and Waterston, Genetics 1984, 108(4):859-877] and arthropods [Jacobsen et al, supra; Barry et al. Genetics 2004, 166(2):823-833; Lampe et al. EMBO J. 1996, 15(19):5470-5479]. In contrast, the biology of Tel/mariner elements in vertebrate cells/genomes is understudied. Despite being present at thousands of copies per genome, there has previously been no evidence of active transposition, nor of transposition-competent Tc1/mariner elements in vertebrate genomes. Instead, active vertebrate transposons have been synthetically created by phylogeny-informed reanimation of inactive elements. The Sleeping Beauty (SB) transposon from teleosts represents the inaugural representative of vertebrate transposon reanimation [Ivies et al. Cell 1997, 91(4):501-510], and has been subsequently engineered to hyperactivity for applications to transpositional transgenesis (TnT) and gene therapy. Additional transposons from amphibians (Frog Prince) and humans (himar1) have been similarly reanimated [Miskey et al. Nucleic Acids Res 2003, 31(23):6873-6881; Miskey et al. Mol. Cell. Biol. 2007, 27(12):4589-4600].

This document is based on the discovery of Passport, a native Tc1 transposon isolated from a fish (Pleuronectes platessa) that is active in cells from a variety of vertebrate tissue sources. Other active Tc1 transposons have not been identified within the native genome of a vertebrate. As described herein, in transposition assays, the Passport transposon system improved stable cellular transgenesis by over 20 fold, has an apparent preference for insertion within genes, and is subject to overexpression inhibition. Unusually, the 5′UTR of the Passport transposon is required for maximal transposition in a manner that depends on the amino terminus of the Passport transposase. Passport elements share features of two related subfamilies of Tc1 transposons, represented by Eagle/Glan and SSTN/Barb, and appear to have a highly restricted phylogenetic distribution. The availability of an active native vertebrate transposon will allow new insight into the mechanisms of transposition, will provide a platform for the exploration of the hypothesized link between mobile genetic elements and vertebrate evolution, and will complement the available genetic tools for the manipulation of vertebrate genomes.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are hereby incorporated herein by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1A-1C A Binary Passport Transposon System Embodiment. A) Two versions of ITR cloning vectors were developed. Both versions contain native Passport transposon (PTn) ITRs as isolated from plaice around small multiple cloning site (enzymes listed in figure). At the termini of the multiple cloning site are outward facing RNA polymerase sites to aid in cloning of transposon junction sequences. Both vectors are cloned into a minimal vector backbone consisting of only the Co1E1 origin of replication (ORI) and kanamycin phosphotransferase (KanR). pPTn1-SE is distinguished from pPTn2-SE by the incorporation of an additional 147 bp cis element that corresponds to sequence found inside of the left ITR within the plaice genome including a portion of the 5′ untranslated region (5′ UTR) of the Passport transposase. B) The Passport transposase was cloned as either a wild-type version (PTs1) or as an alternate version (PTs2) including a glycine residue inserted as the second amino acid; the sequences depict the location of the additional Glycine inserted into PTs2 (SEQ ID NOs: 1-4). C) Both versions of Passport protein coding sequences were cloned into expression vectors utilizing the human Ubiquitin C promoter region (Ub) or the hybrid mCAGs promoter (mCAG) to drive expression of transposase.

FIG. 2—Passport functions in human cells. An expression cassette that yields G418 resistance (GFP-IRES-Neo) was cloned into either pPTn1 or pPTn2 that differ by the inclusion of a 147 bp cis element within pPTn1. Both of these vectors were transfected into HT1080 cells along with pKC-PTs1, pKC-PTs2, or pCMV-Bgal (a no transposase control). After selection in G418, stable colonies were stained and counted. The average number of colonies is graphed and shown with the standard error. The average number of colonies was 50.3+/−10.3 (N=12), 26.2+/−10.1 (N=10), 38.8+/−9.8 (N=10), 32.7+/−16.9 (N=6), 2.7+/−0.97 (N=15), and 1.4+/−0.7 (N=5) for pKC-PTs1 with pPTn1P, pKC-PTs1 with pPTn2P, pKC-PTs2 with pPTn1P, pKC-PTs2 with pPTn2P, pCMV-Bgal with pPTn1P, and pCMV-Bgal with pPTn2P, respectively. The significance of the addition of transposase pTs1 or pTs2 with the pPTn1P transposons compared with addition of beta-galactosidase was measured using a one-tailed paired t-test (p<0.0001). In the case where pPTn2P was used, the p-values were <0.06. Differences observed using the native transposase (pTs1) with the 147 bp cis element in pPTn1P versus the transposons with ITRs only in pPTn2P showed a significant increase in transposition with a p-value of 0.06.

FIG. 3A-3C Examination of overexpression inhibition. A) To examine the effect of transposase dose on transposition rates, a constant amount of pTnP-GeN (75 femtomoles) was co-transfected with 5 different molar ratios of transposase expression vector driven by either the human Ubiquitin C promoter (pKUb-Ts) or the mCAGs promoter (pKC-Ts), where T and Ts generically refer to either SB or Passport components. In all cases the total amount of DNA transfected was adjusted to 2 μg by the addition of the appropriate amount of pCMV-Bgal. After transfection and selection in G418, colonies were counted and the data compared to an internal reference transfection of SB at a ratio of 1:1 U. The raw data for the internal reference transfection came from a total of 30 replicates and ranged from 68 to 324, with a median of 150 and a mean of 170 (data not shown). The relative transposition efficiencies confirm overexpression inhibition of B) the SB transposon system and C) demonstrate overexpression inhibition of the Passport transposon system. Error bars represent the standard error.

FIG. 4A-4C Passport functions in cells from a wide variety of vertebrate sources. A) A Passport transposon that expresses Puromycin phosphotransferase was co-transfected with a source of Passport transposase, pKC-PTs1 (+PTs) or pCMV-Bgal (−PTs). Cells were selected in puromycin and stable colonies were counted. B) HeLa, CHO, Vero, and HT1080 cells displayed an increase in stable colony formation with the addition of Passport transposase (p=1.08 e-9, 0.070, 0.031, and 0.0001, respectively). C) 3T3, TT, DF1, and PEGE cells produced less stable colonies under these transfection conditions; however, the addition of Passport transposase significantly improved colony formation (p=0.005, 0.0003, 0.003, 0.004, respectively). P-values are based on a one-tailed, unpaired t-test.

FIG. 5A-5C Evaluation of diversity and number Passport genomic integrations. A) TnT mediated recombination into the genome should result in recognition) of variable length fragments following hybridization of a PTK probe (red bar) to genomic DNA digested with AseI. The sizes of the fragments are dependent on the proximity of AseI recognition sites in the neighboring chromatin. B) Commonly, when DNA integrates without the enzymatic activity of transposase, head to tail concatemers of variable length are formed and integrate into the genome by non-homologous end joining. In this case, the size of this internal high-representative fragment (˜5.1 kb) is predictable based on the location of AseI sites within the transposon donor plasmid. C) Southern hybridization of 15 independent HT1080 clones. The paired head to tail arrows indicate the expected position of pPTnP-PTK concatemers formed during integration by non-homologous end joining. The line with outward facing arrowheads represents the size of the transposon and therefore the minimal expected size of a hybridizing fragment integrated by TnT. The asterisks mark two bands present in the HT1080 DNA that hybridize weakly with the PTK probe used here.

FIG. 6A-6B Phylogeny of Passport-like transposons and their hosts. A) Neighbor joining plot of multiply aligned transposase consensus amino acid sequences. Sequences were aligned with ClustalW, and plotted with NJplot. Numbers represent the percentage frequencies with which the tree topology was returned after 1000 iterations. The tree is rooted to Tc1 from C. elegans. Transposon designation is prefixed by host species identifier; om, rainbow trout; ol, medaka; ga, stickleback; tr, pufferfish; ss, Atlantic salmon; ce, C. elegans; rt, Rana temporaria (frog); xt, Xenopus tropicalis. Passport, Frog Prince, and Sleeping Beauty were isolated from Pleuronectes platessa, Rana sylvestris, and a variety of salmonid species, respectively. B) Phylogeny of host species adapted from Nelson 2006 [37]. The colored dots assist in pairing the transposons shown in A with the species from B.

FIG. 7A-7B Comparison of repeat sequences and transposase DNA-binding domains of Passport-like transposons. A) Comparison of terminal and internal 5′ repeats. Host species identifier as for FIG. 6 prefixes transposon designation (SEQ ID NOs: 5-20). Bars below the line indicate the conserved repeat units from within the inverted repeats. Highlighted sequences delineate differences between the Eagle/Glan and SSTN/Barb families. For the ITR sequences, Passport clearly aligns more closely with the SSTN/Barb families within the direct repeats, but evidence of convergence towards Eagle/Glan sequences are observed just outside of these direct repeats as indicated the asterisks. B) Comparison of the putative DNA-binding domains of Passport and related transposases (SEQ ID NOs: 21-28). Shaded residues marked with open-ended arrows (>) indicate the amino acids that distinguish members of the Eagle/Glan family from SSTN/Barb. Shaded residues marked with closed-ended arrows indicate the convergence of the active Passport sequence towards the Eagle/Glan family to which it is more closely related over the length of the entire protein, whereas residues shaded and marked with unfilled block arrows show some convergence of the X. tropicalis Eagle element towards the SSTN/Barb subfamily. Residues SL shaded and boxed seem to be unique within Passport.

FIG. 8A-8B is an alignment of the amino acid sequence of the Passport (PPTs1), Sleeping Beauty 11 (SB11), and Frog Prince (FP) transposases (FIG. 8B, SEQ ID NOs: 29-32). The percent identity/similarity between the three transposases is shown on top (FIG. 8A).

FIG. 9 Integration sequence preferences of the Passport transposase. The results from 27 insertion sites are graphically represented, wherein the height of each indicated base is proportional to the relative conservation of sequence among integration sites.

FIG. 10A-10F contains sequence of the Passport transposon. The first sequence is PPTN4 (SEQ ID NO: 33), published by Leaver in 2001 (Gene, 271(2):203-214). FIG. 10B is PTs1 (SEQ ID NO: 29) and is the amino acid sequence of the native Passport transposase as found in PPTN4. FIG. 10C shows PTn1_ITR(L) (SEQ ID NO: 34) and FIG. 10D shows PTn1_ITR(R) (SEQ ID NO: 35, which are sequences used in PTn1. The sequences in FIGS. 10E-F are the IR/DR sequences (SEQ ID NOs: 36-37).

### DETAILED DESCRIPTION

The present document relates to a Plaice transposon system termed “Passport” that can be used to introduce nucleic acid sequences into the DNA of a cell or embryo. Transposons are mobile, in that they can move from one position on DNA to a second position on DNA in the presence of a transposase. Passport transposons are a viable way of introducing DNA into a cell and can be used to modify germline and somatic cells for the production of transgenic animals, germline mutagenesis, or for somatic modification like gene therapy. As described herein, the native Passport transposon was domesticated as a binary nonautonomous system, and have demonstrated cis (IR/DRs) and trans (transposase) acting components that when combined are competent for transposition. In addition, a 20 to 40-fold increase in transgenesis was observed in vitro (HeLa cells and HT1080 cells) using the Passport transposon. This level of improvement is significant and is similar to levels observed with SB when it was initially reanimated (pT with SB10). An understanding of the interactions between the transposase DNA binding domains and their cognate transposon ITR sequences is critical for deriving an element with sufficient transpositonal efficiency for widespread use as a molecular genetic tool. The comparison of Passport and Eagle ITR/transposase DNA binding domains indicates that there are sequence residues specific to each element.

A significant preference was observed for integration into genes (likelihood ratio>5000:1), suggesting divergence in the mechanism for integration site selection amongst vertebrate Tel transposons. This characteristic has been observed for the piggyBac transposon system, a non-Tel element, but contrasts sharply with the more random integration site preferences for the SB transposon system, suggesting that Passport may be especially suitable for functional genomics applications that rely on insertional mutagenesis.

Regardless of the inherent differences in transposition of Tel-like vertebrate transposons like Passport and SB, the availability of multiple transposon systems for genetic manipulation is beneficial. There are a variety of other transposons now capable of transposition in vertebrate cells, including SB (see U.S. Pat. No. 6,489,458, U.S. Pat. No. 6,613,752), Frog Prince (see US US20050241007), To12 [Hori et al. J. Marine Biotechnology 1998, 6(4):206-207] see US20050177890, U.S. Pat. No. 7,034,115, minos [Klinakis et al. EMBO Rep 2000, 1(5):416-421; Franz and Savakis Nucleic Acids Res 1991, 19(23):6646.], piggyBac [Ding et al. Cell 2005, 122(3):473-483; Fraser et al. Insect Mol Biol 1996, 5(2):141-151] (see US20090042297, US20070204356), Ac/Ds [Emelyanov et al. Genetics 2006, 174(3):1095-1104.], Tol1 [Koga et al. J. Human Genetics 2007, 52(7):628-635], HsMar1 [Miskey et al. Mol Cell. Biol. 2007, 27(12):4589-4600], and Harbinger [Sinzelle et al. Proc. Natl. Acad. Sci. USA 2008, 105(12):4715-4720], see U.S. Pat. Nos. See also Clar et al., Nucleic Acids Research, 2009, 37(4): 1239-1247 which describes the Passport system, which article is hereby incorporated by reference herein in its entirety. The application of multiple transposon systems in a serial manner could allow the production of stable multi-transgene containing animals without the chance of remobilizing previously integrated transposons. The parallel use of multiple transposon systems may overcome some aspects of overproduction inhibition permitting more efficient TnT or gene therapy or increasing the saturation of mutagenesis screening by taking advantage of differences in integration site preferences.

### Nucleic Acids and Nucleic Acid Constructs

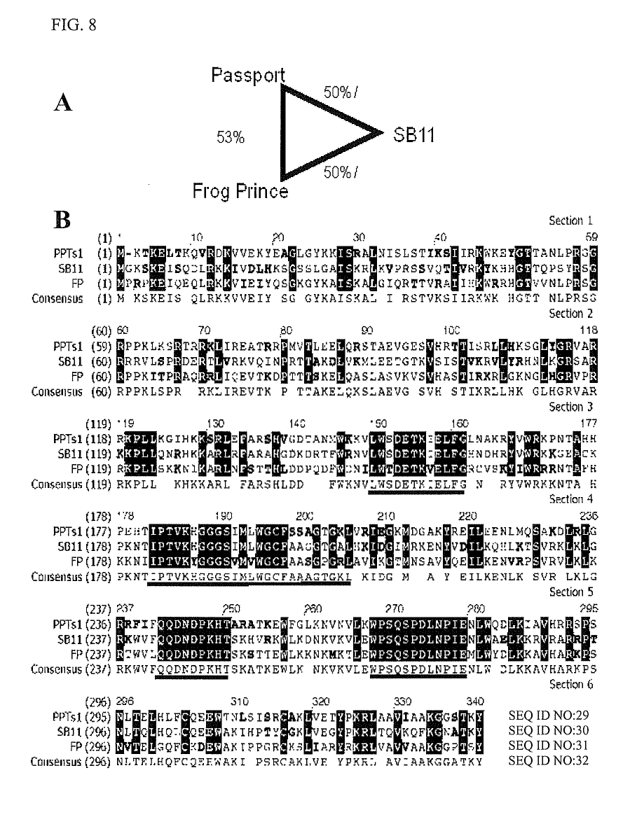

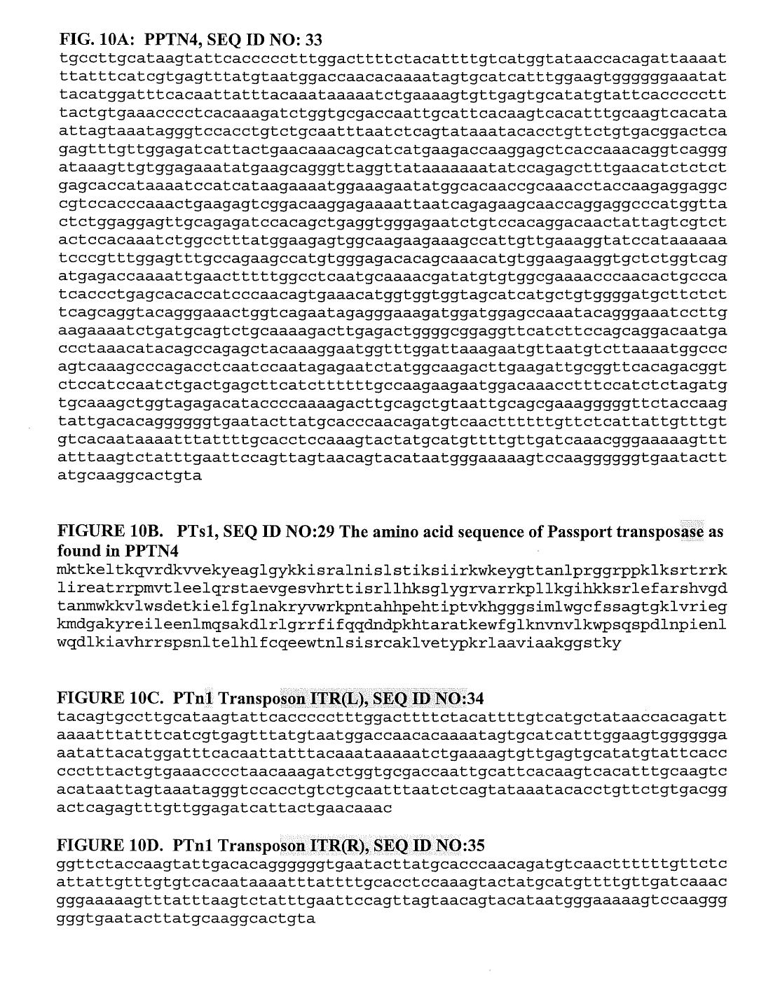

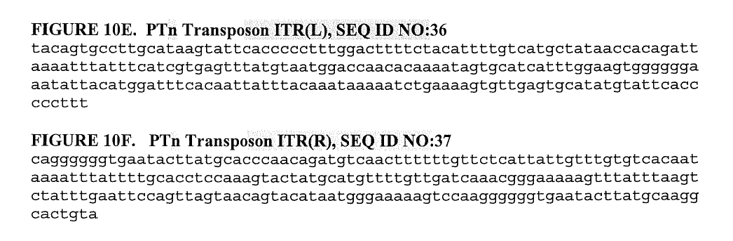

This document provides nucleic acid molecules that encode transposase polypeptides and nucleic acid constructs containing the same. A transposase is an enzyme that is capable of binding to inverted repeats of a transposon and catalyzes the incorporation of the transposon into DNA. The Passport transposase sequence is shown in FIG. 8 (PPTs1) and FIG. 10 (PTs1). This document also provides nucleic acid constructs that contain a transcriptional unit flanked by inverted repeats of the Passport transposon. Nucleic acid constructs containing such a transcriptional unit can be used in combination with a source of a transposase to introduce a target DNA into a host chromosome. A transposase can be encoded on the same nucleic acid construct as the target nucleic acid, can be introduced on a separate nucleic acid construct, or provided as an mRNA (e.g., an in vitro transcribed and capped mRNA).

The term “nucleic acid” as used herein encompasses both RNA and DNA, including cDNA, genomic DNA, and synthetic (e.g., chemically synthesized) DNA. A nucleic acid can be double-stranded or single-stranded. A single-stranded nucleic acid can be the sense strand or the antisense strand. In addition, a nucleic acid can be circular or linear.

An “isolated nucleic acid” refers to a nucleic acid that is separated from other nucleic acid molecules that are present in a naturally-occurring genome, including nucleic acids that normally flank one or both sides of the nucleic acid in the naturally-occurring genome. The term “isolated” as used herein with respect to nucleic acids also includes any non-naturally-occurring nucleic acid sequence, since such non-naturally-occurring sequences are not found in nature and do not have immediately contiguous sequences in a naturally-occurring genome.

An isolated nucleic acid can be, for example, a DNA molecule, provided one of the nucleic acid sequences normally found immediately flanking that DNA molecule in a naturally-occurring genome is removed or absent. Thus, an isolated nucleic acid includes, without limitation, a DNA molecule that exists as a separate molecule (e.g., a chemically synthesized nucleic acid, or a cDNA or genomic DNA fragment produced by PCR or restriction endonuclease treatment) independent of other sequences as well as DNA that is incorporated into a vector, an autonomously replicating plasmid, a virus (e.g., any paramyxovirus, retrovirus, lentivirus, adenovirus, or herpes virus), or into the genomic DNA of a prokaryote or eukaryote. In addition, an isolated nucleic acid can include an engineered nucleic acid such as a DNA molecule that is part of a hybrid or fusion nucleic acid. A nucleic acid existing among hundreds to millions of other nucleic acids within, for example, cDNA libraries or genomic libraries, or gel slices containing a genomic DNA restriction digest, is not considered an isolated nucleic acid.

The term “transposase polypeptide” as used herein refers to any amino acid sequence that is at least 70 percent (e.g., at least 75, 80, 85, 90, 95, 99, or 100 percent) identical to the PPTs1 sequence set forth in FIG. 8. The percent identity between a particular amino acid sequence and the PPTs1 amino acid sequence set forth in FIG. 8 is determined as follows. First, the amino acid sequences are aligned using the BLAST 2 Sequences (Bl2seq) program from the stand-alone version of BLASTZ containing BLASTP version 2.0.14. This stand-alone version of BLASTZ can be obtained from Fish & Richardson's web site (e.g., www.fr.com/blast/) or the U.S. government's National Center for Biotechnology Information web site (www.ncbi.nlm nih.gov). Instructions explaining how to use the Bl2seq program can be found in the readme file accompanying BLASTZ. Bl2seq performs a comparison between two amino acid sequences using the BLASTP algorithm. To compare two amino acid sequences, the options of Bl2seq are set as follows: -i is set to a file containing the first amino acid sequence to be compared (e.g., C:\seql.txt); -j is set to a file containing the second amino acid sequence to be compared (e.g., C:\seq2.txt); -p is set to blastp; -o is set to any desired file name (e.g., C:\output.txt); and all other options are left at their default setting. For example, the following command can be used to generate an output file containing a comparison between two amino acid sequences: C:\B12seq-i c:\seq1.txt-j c:\seq2.txt-p blastp-o c:\output.txt. If the two compared sequences share homology, then the designated output file will present those regions of homology as aligned sequences. If the two compared sequences do not share homology, then the designated output file will not present aligned sequences.

Once aligned, the number of matches is determined by counting the number of positions where an identical amino acid residue is presented in both sequences. The percent identity is determined by dividing the number of matches by the length of the full-length transposase polypeptide amino acid sequence followed by multiplying the resulting value by 100.

It is noted that the percent identity value is rounded to the nearest tenth. For example, 78.11, 78.12, 78.13, and 78.14 is rounded down to 78.1, while 78.15, 78.16, 78.17, 78.18, and 78.19 is rounded up to 78.2. It also is noted that the length value will always be an integer.

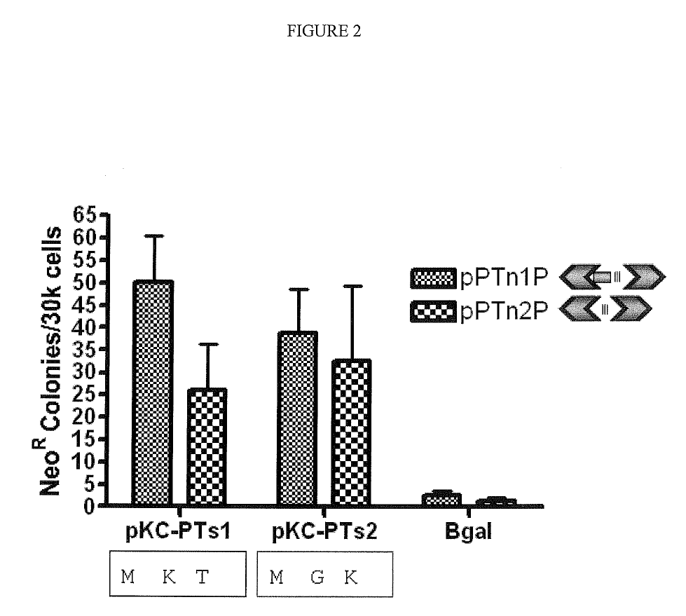

A nucleic acid molecule described herein can encode a transposase that has a mutation relative to the amino acid sequence set forth in FIG. 8 and FIG. 10. Possible mutations include, without limitation, substitutions (e.g., transitions and transversions), deletions, insertions, and combinations of substitutions, deletions, and insertions. Nucleic acid molecules can include a single nucleotide mutation or more than one mutation, or more than one type of mutation. For example, a nucleic acid molecule encoding the Passport transposase can be modified such that a glycine residue is encoded after the initial methionine. See e.g., FIG. 2. Nucleic acids can be modified using common molecular cloning techniques (e.g., site-directed mutagenesis) to generate mutations. Polymerase chain reaction (PCR) and nucleic acid hybridization techniques can be used to identify nucleic acids encoding transposase polypeptides having altered amino acid sequences.

Transposases may be created by derivitizing sequences set forth herein and testing them for specific binding and/or mobilization of target nucleic acid in combination with transposons as described herein, e.g., transposons with that have SEQ ID NOs:34 and 35. Similarly, transposons may be created by derivitizing sequences set forth herein and testing them for specific binding and/or mobilization of target nucleic acid in combination with transposases with a sequence as described herein, e.g., SEQ ID NO:29.

Specific binding, as that term is commonly used in the biological arts, generally refers to a molecule that binds to a target with a relatively high affinity compared to non-targets, and generally involves a plurality of non-covalent interactions, such as electrostatic interactions, van der Waals interactions, hydrogen bonding, and the like. Specific binding interactions characterize antibody-antigen binding, enzyme-substrate binding, and binding between transposases and inverted terminal repeats of transposons. While molecules may transiently interact with molecules besides their targets from time to time, such binding is said to lack specificity and is not specific binding. One feature that distinguishes transposases from each other is that they do not specifically bind to transposons recognized by other transposases. FIG. 8 depicts identity of Passport with the closest known transposases, SB11 and Frog Prince; as is evident, an identity of more than about 70% or 80% is more than adequate to establish that the transposase has a Passport family member structure. Further or alternative establishment of the transposon or transposase structure may be achieved by testing the increase in transgenesis in vitro (for instance, with HeLa cells and/or HT1080 cells), with a criterion being a more than 10-fold to 40-fold increase; artisans will immediately appreciate that all the ranges and values within the explicitly stated ranges are contemplated.

Embodiments thus include transposases with 70%-99% identity to SEQ ID NO:29, with the transposases binding to a transposon that has one or both of SEQ ID NOs: 34 and 35. And embodiments include transposons with 70%-99% identity to SEQ ID NO:34 and/or 35, with the transposons binding to a transposase that has SEQ ID NO:29. The transposons may have intervening nucleic acids between the inverted terminal repeats so that identity should be compared across suitably aligned sections. Artisans will immediately appreciate that all the ranges and values within the explicitly stated ranges of 70%-99% are contemplated, e.g., at least 80%, at least 85%, at least 95%.

Nucleic acid molecules can be obtained using any method including, without limitation, common molecular cloning and chemical nucleic acid synthesis techniques. For example, PCR can be used to construct nucleic acid molecules that encode transposases. PCR refers to a procedure or technique in which target nucleic acid is amplified in a manner similar to that described in U.S. Pat. No. 4,683,195, and subsequent modifications of the procedure described therein.

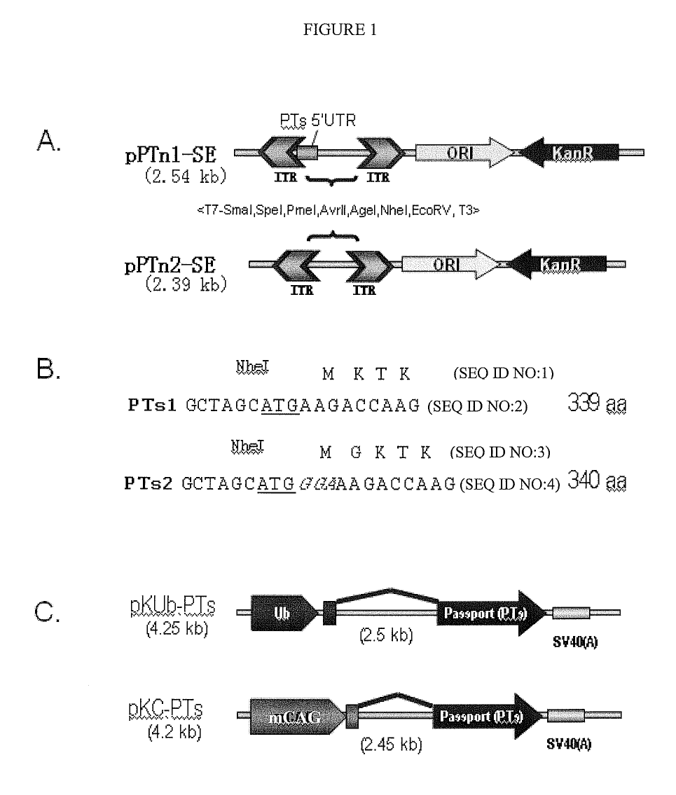

In transposon systems for transpositional transgenesis, at least one nucleic acid construct is used that includes a transcriptional unit, i.e., a regulatory region operably linked to a target nucleic acid sequence, flanked by an inverted repeat of a transposon. For example, the inverted repeats of a transposon can have at least 70% sequence identity (e.g., at least 75%, 80%, 85%, 90%, 95%, 99% or 100%) to the nucleotide sequences set forth in FIG. 10 (e.g., PTn_ITR(L) and PTn_ITR(R). FIG. 10 also contains the complete nucleotide sequence of a Passport transposon. In addition, a nucleic acid construct can include the 5′ untranslated region (UTR) of the Passport transposase, e.g., as set forth in FIG. 1A.

Insulator elements also can be included in a nucleic acid construct to maintain expression of the target nucleic acid and to inhibit the unwanted transcription of host genes. See, for example, U.S. Patent Publication No. 20040203158. Typically, an insulator element flanks each side of the transcriptional unit and is internal to the inverted repeat of the transposon. Non-limiting examples of insulator elements include the matrix attachment region (MAR) type insulator elements and border-type insulator elements. See, for example, U.S. Pat. Nos. 6,395,549, 5,731,178, 6,100,448, and 5,610,053, and U.S. Patent Publication No. 20040203158.

Nucleic acid constructs described herein can be used to introduce a target nucleic acid into a cell or to produce transgenic animal. As used herein, the term “nucleic acid” includes DNA, RNA, and nucleic acid analogs, and nucleic acids that are double-stranded or single-stranded (i.e., a sense or an antisense single strand). Nucleic acid analogs can be modified at the base moiety, sugar moiety, or phosphate backbone to improve, for example, stability, hybridization, or solubility of the nucleic acid. Modifications at the base moiety include deoxyuridine for deoxythymidine, and 5-methyl-2′-deoxycytidine and 5-bromo-2′-doxycytidine for deoxycytidine. Modifications of the sugar moiety include modification of the 2′ hydroxyl of the ribose sugar to form 2′-O-methyl or 2′-O-allyl sugars. The deoxyribose phosphate backbone can be modified to produce morpholino nucleic acids, in which each base moiety is linked to a six membered, morpholino ring, or peptide nucleic acids, in which the deoxyphosphate backbone is replaced by a pseudopeptide backbone and the four bases are retained. See, Summerton and Weller (1997) Antisense Nucleic Acid Drug Dev. 7(3):187-195; and Hyrup et al. (1996) Bioorgan. Med. Chem. 4(1):5-23. In addition, the deoxyphosphate backbone can be replaced with, for example, a phosphorothioate or phosphorodithioate backbone, a phosphoroamidite, or an alkyl phosphotriester backbone.

The target nucleic acid sequence can be operably linked to a regulatory region such as a promoter. Regulatory regions can be porcine regulatory regions or can be from other species, including humans, monkeys, hamsters, mice, chickens, and turkeys. As used herein, “operably linked” refers to positioning of a regulatory region relative to a nucleic acid sequence in such a way as to permit or facilitate transcription of the target nucleic acid.

Any type of promoter can be operably linked to a target nucleic acid sequence. Examples of promoters include, without limitation, tissue-specific promoters, constitutive promoters, and promoters responsive or unresponsive to a particular stimulus. Suitable tissue specific promoters can result in preferential expression of a nucleic acid transcript in ∂ cells and include, for example, the human insulin promoter. Other tissue specific promoters can result in preferential expression in, for example, hepatocytes or heart tissue and can include the albumin or alpha-myosin heavy chain promoters, respectively.

In other embodiments, a promoter that facilitates the expression of a nucleic acid molecule without significant tissue- or temporal-specificity can be used (i.e., a constitutive promoter). For example, a beta-actin promoter such as the chicken ∂-actin gene promoter, ubiquitin promoter, miniCAGs promoter, glyceraldehyde-3-phosphate dehydrogenase (GAPDH) promoter, or 3-phosphoglycerate kinase (PGK) promoter can be used, as well as viral promoters such as the herpes virus thymidine kinase (TK) promoter, the SV40 promoter, or a cytomegalovirus (CMV) promoter. In some embodiments, a fusion of the chicken 4 actin gene promoter and the CMV enhancer is used as a promoter. See, for example, Xu et al. (2001) Hum. Gene Ther. 12(5):563-73; and Kiwaki et al. (1996) Hum. Gene Ther. 7(7):821-30.

An example of an inducible promoter is the tetracycline (tet)-on promoter system, which can be used to regulate transcription of the nucleic acid. In this system, a mutated Tet repressor (TetR) is fused to the activation domain of herpes simplex VP 16 (transactivator protein) to create a tetracycline-controlled transcriptional activator (tTA), which is regulated by tet or doxycycline (dox). In the absence of antibiotic, transcription is minimal, while in the presence of tet or dox, transcription is induced. Alternative inducible systems include the ecdysone or rapamycin systems. Ecdysone is an insect molting hormone whose production is controlled by a heterodimer of the ecdysone receptor and the product of the ultraspiracle gene (USP). Expression is induced by treatment with ecdysone or an analog of ecdysone such as muristerone A.

Additional regulatory regions that may be useful in nucleic acid constructs, include, but are not limited to, polyadenylation sequences, translation control sequences (e.g., an internal ribosome entry segment, IRES), enhancers, inducible elements, or introns. Such regulatory regions may not be necessary, although they may increase expression by affecting transcription, stability of the mRNA, translational efficiency, or the like. Such regulatory regions can be included in a nucleic acid construct as desired to obtain optimal expression of the nucleic acids in the cell(s). Sufficient expression, however, can sometimes be obtained without such additional elements.

Other elements that can be included on a nucleic acid construct encode signal peptides or selectable markers. Signal peptides can be used such that an encoded polypeptide is directed to a particular cellular location (e.g., the cell surface). Non-limiting examples of selectable markers include puromycin, adenosine deaminase (ADA), aminoglycoside phosphotransferase (neo, G418, APH), dihydrofolate reductase (DHFR), hygromycin-B-phosphtransferase, thymidine kinase (TK), and xanthin-guanine phosphoribosyltransferase (XGPRT). Such markers are useful for selecting stable transformants in culture. Other selectable markers include fluorescent polypeptides, such as green fluorescent protein or yellow fluorescent protein.

In some embodiments, a sequence encoding a selectable marker can be flanked by recognition sequences for a recombinase such as, e.g., Cre or Flp. For example, the selectable marker can be flanked by loxP recognition sites (34 by recognition sites recognized by the Cre recombinase) or FRT recognition sites such that the selectable marker can be excised from the construct. See, Orban, et al., Proc. Natl. Acad. Sci. (1992) 89 (15): 6861-6865, for a review of Cre/lox technology, and Brand and Dymecki, Dev. Cell (2004) 6(1):7-28.

In some embodiments, the target nucleic acid encodes a polypeptide. A nucleic acid sequence encoding a polypeptide can include a tag sequence that encodes a “tag” designed to facilitate subsequent manipulation of the encoded polypeptide (e.g., to facilitate localization or detection). Tag sequences can be inserted in the nucleic acid sequence encoding the polypeptide such that the encoded tag is located at either the carboxyl or amino terminus of the polypeptide. Non-limiting examples of encoded tags include glutathione S-transferase (GST) and Flag™ tag (Kodak, New Haven, Conn.).

In other embodiments, the target nucleic acid sequence induces RNA interference against a target nucleic acid such that expression of the target nucleic acid is reduced. Constructs for siRNA can be produced as described, for example, in Fire et al. (1998) Nature 391:806-811; Romano and Masino (1992) Mol. Microbial. 6:3343-3353; Cogoni et al. (1996) EMBO J. 15:3153-3163; Cogoni and Masino (1999) Nature 399:166-169; Misquitta and Paterson (1999) Proc. Natl. Acad. Sci. USA 96:1451-1456; and Kennerdell and Carthew (1998) Cell 95:1017-1026. Constructs for shRNA can be produced as described by McIntyre and Fanning (2006) BMC Biotechnology 6:1. In general, shRNAs are transcribed as a single-stranded RNA molecule containing complementary regions, which can anneal and form short hairpins. Embodiments include methods and materials as set forth in copending U.S. Ser. No. 61/081,293 filed Jul. 16, 2008 and U.S. Ser. No. ______ by Fahrenkrug et al. filed Jul. 16, 2009, which are hereby incorporated by reference herein, for example, by using transposons and/or transposases as set forth herein being used to introduce target nucleic acids or create transgenic cells or animals therein described.

In some embodiments, a nucleic acid construct can be methylated using an SssI CpG methylase (New England Biolabs, Ipswich, Mass.). In general, a nucleic acid construct can be incubated with S-adenosylmethionine and SssI CpG-methylase in buffer at 37° C. Hypermethylation can be confirmed by incubating the construct with one unit of HinP1I endonuclease for 1 hour at 37° C. and assaying by agarose gel electrophoresis.

Nucleic acid constructs described herein can be introduced into embryonic, fetal, or adult cells of any type, including, for example, germ cells such as an oocyte or an egg, a progenitor cell, an adult or embryonic stem cell, a kidney cell such as a PK-15 cell, an islet cell, a beta cell, a liver cell, or a fibroblast such as a dermal fibroblast, using a variety of techniques.

### Polypeptides

This document also provides transposase polypeptides. As used here, a “polypeptide” refers to a chain of amino acid residues, regardless of post-translational modification (e.g., phosphorylation or glycosylation). A transposase polypeptide described herein has an amino acid sequence that is at least 50 percent (e.g., at least 55, 60, 65, 70, 75, 80, 85, 90, 95, 99, or 100 percent) identical to the PPTs sequence set forth in SEQ ID NO:29.

Transposase polypeptides described herein can include at least one amino acid substitution relative to the amino acid sequence of SEQ ID NO:29. Amino acid substitutions can be conservative or non-conservative. Conservative amino acid substitutions replace an amino acid with an amino acid of the same class, whereas non-conservative amino acid substitutions replace an amino acid with an amino acid of a different class. Examples of conservative substitutions include amino acid substitutions within the following groups: (1) glycine and alanine; (2) valine, isoleucine, and leucine; (3) aspartic acid and glutamic acid; (4) asparagine, glutamine, serine, and threonine; (5) lysine, histidine, and arginine; and (6) phenylalanine and tyrosine.

Non-conservative amino acid substitutions may replace an amino acid of one class with an amino acid of a different class. Non-conservative substitutions can make a substantial change in the charge or hydrophobicity of the gene product. Non-conservative amino acid substitutions also can make a substantial change in the bulk of the residue side chain, e.g., substituting an alanine residue for an isoleucine residue. Examples of non-conservative substitutions include the substitution of a basic amino acid for a non-polar amino acid or a polar amino acid for an acidic amino acid.

Transposase polypeptides can be produced using any method. For example, transposase polypeptides can be produced by chemical synthesis. Alternatively, transposase polypeptides described herein can be produced by standard recombinant technology using heterologous expression vectors encoding transposase polypeptides. Expression vectors can be introduced into host cells (e.g., by transformation or transfection) for expression of the encoded polypeptide, which then can be purified. Expression systems that can be used for small or large scale production of transposase polypeptides include, without limitation, microorganisms such as bacteria (e.g., E. coli and B. subtilis) transformed with recombinant bacteriophage DNA, plasmid DNA, or cosmid DNA expression vectors containing the nucleic acid molecules described herein, and yeast (e.g., S. cerevisiae) transformed with recombinant yeast expression vectors containing the nucleic acid molecules described herein. Useful expression systems also include insect cell systems infected with recombinant virus expression vectors (e.g., baculovirus) containing the nucleic acid molecules of the invention, and plant cell systems infected with recombinant virus expression vectors (e.g., tobacco mosaic virus) or transformed with recombinant plasmid expression vectors (e.g., Ti plasmid) containing the nucleic acid molecules described herein. Transposase polypeptides also can be produced using mammalian expression systems, which include cells (e.g., primary cells or immortalized cell lines such as COS cells, Chinese hamster ovary cells, HeLa cells, human embryonic kidney 293 cells, and 3T3 μl cells) harboring recombinant expression constructs containing promoters derived from the genome of mammalian cells (e.g., the metallothionein promoter) or from mammalian viruses (e.g., the adenovirus late promoter and the cytomegalovirus promoter), along with the nucleic acids described herein.

### Transfection

Cells may be transfected in vitro or in vivo. Ex vivo cellular transfection refers to transfection of cells in vitro and subsequent introduction into a patient (human or animal). Autologous cells may be transfected in vitro or ex vivo, meaning that the transfected cells are from the patient that receives the transfected cells. Similarly, allogeneic or xenogeneic cells may be transfected.

A transposon and a transposase may be introduced at the same time, or sequentially in time, and on the same vehicle, or separately, e.g., on the same plasmid, on separate plasmids, or one in a particle or liposome and another via a vector. For example, both the transposon and the transposase gene can be contained together on the same recombinant viral genome; a single infection delivers both parts of the SB system such that expression of the transposase then directs cleavage of the transposon from the recombinant viral genome for subsequent integration into a cellular chromosome. In another example, the transposase and the transposon can be delivered separately by a combination of viruses and/or non-viral systems such as lipid-containing reagents. In these cases either the transposon and/or the transposase gene can be delivered by a recombinant virus. The expressed transposase gene directs liberation of the transposon from its carrier DNA (viral genome) for integration into chromosomal DNA. Delivery of a transposase as RNA (e.g., mRNA) provides a burst of activity followed by degradation of the transposase, with the RNA not becoming incorporated into the patient's genome.

In one embodiment, the transposase is provided to the cell as a protein and in another the transposase is provided to the cell as nucleic acid encoding the protein. In one embodiment the nucleic acid is RNA and in another the nucleic acid is DNA. In yet another embodiment, the nucleic acid encoding the transposase is integrated into the genome of the cell. The nucleic acid fragment can be, e.g., part of a plasmid or a recombinant viral vector. Further, nucleic acid encoding the protein can be incorporated into a cell through a viral vector, cationic lipid, or other transfection mechanisms including electroporation or particle bombardment used for eukaryotic cells.

A nucleic acid fragment can be introduced into the cell as a linear fragment or as a circularized fragment, e.g, as a plasmid or as recombinant viral DNA. The nucleic acid sequence may comprise at least a portion of an open reading frame to produce an amino-acid containing product. The transposase protein can be introduced into the cell as ribonucleic acid, including mRNA; as DNA present in the cell as extrachromosomal DNA including, but not limited to, episomal DNA, as plasmid DNA, or as viral nucleic acid. Further, DNA encoding the protein can be integrated into the genome of the cell for constitutive or inducible expression. Where the protein is introduced into the cell as nucleic acid, the protein encoding sequence may optionally be operably linked to a promoter.

Another embodiment of the invention relates to a method for identifying a gene in a genome of a cell. For instance a method may be used involving introducing a nucleic acid fragment and a transposase protein into a cell, wherein the nucleic acid fragment comprises a nucleic acid sequence positioned between at least two inverted repeats into a cell wherein the inverted repeats can bind to the transposase protein and wherein the nucleic acid fragment is capable of integrating into DNA in a cell in the presence of the transposase protein; digesting the DNA of the cell with a restriction endonuclease capable of cleaving the nucleic acid sequence; identifying the inverted repeat sequences; sequencing the nucleic acid close to the inverted repeat sequences; and comparing the DNA sequence with sequence information in a computer database.

### Vectors

Nucleic acids can be incorporated into vectors. Vectors most often contain one or more expression cassettes that comprise one or more expression control sequences, wherein an expression control sequence is a DNA sequence that controls and regulates the transcription and/or translation of another DNA sequence or mRNA, respectively. Expression control sequences include, for example, promoter sequences, transcriptional enhancer elements, start codons, stop codons, and any other nucleic acid elements required for RNA polymerase binding, initiation, or termination of transcription. A wide range of expression control sequences is well known in the art and is commercially available. A transcriptional unit in a vector may thus comprise an expression control sequence operably linked to an exogenous nucleic acid sequence. For example, a DNA sequence is operably linked to an expression-control sequence, such as a promoter when the expression control sequence controls and regulates the transcription and translation of that DNA sequence. Examples of vectors include: plasmids (which may also be a carrier of another type of vector), adenovirus, adeno-associated virus (AAV), lentivirus (e.g., modified HIV-1, SIV or FIV), retrovirus (e.g., ASV, ALV or MoMLV), and transposons (e.g., Sleeping Beauty, P-elements, Tol-2, Frog Prince, piggyBac).

### Pharmaceutically Acceptable Carriers and Administration

The transposases and/or transposons may be prepared in combination with a pharmaceutically acceptable carrier and/or suitably administered. One aspect of employing a non-viral vector, e.g., a transposon system, is the mechanism for its delivery. One method for transposons is the hydrodynamic delivery wherein a relatively large volume of transgenic DNA is injected into the circulatory system (the tail vein in mice) under high pressure—most of this DNA winds up in cells of the liver. Another method is to use negatively charged liposomes containing galactocerebroside, or complexed with polyethyleneimine (PEI), which may be complexed with ligands such as lactose or galactose for tissue-specific uptake and which have been effective in delivering nucleic acids into hepatoma cells, primary hepatocytes and liver and lung cells in living mice.

The delivery of transposons, in plasmid carrier molecules, to any tissue in the body is contemplated, including cells found in blood, liver, lung, pancreas, muscle, eye, brain, nervous system, organs, dermis, epidermis, cardiac, and vasculature. For example delivery may be by, direct injection into or near the desired tissue, complexation with molecules that preferentially or specifically bind to a target in the desired tissue, control release, oral, intramuscular, and other delivery systems that are known to those skilled in these arts.

Examples of delivery of certain embodiments herein include via injection, such as intravenously, intramuscularly, or subcutaneously, and in a pharmaceutically acceptable carriers, e.g., in solution and sterile vehicles, such as physiological buffers (e.g., saline solution or glucose serum). The embodiments may also be administered orally or rectally, when they are combined with pharmaceutically acceptable solid or liquid excipients. Embodiments can also be administered externally, for example, in the form of an aerosol with a suitable vehicle suitable for this mode of administration, for example, nasally. Further, delivery through a catheter or other surgical tubing is possible. Alternative routes include tablets, capsules, and the like, nebulizers for liquid formulations, and inhalers for lyophilized or aerosolized agents.

Presently known methods for delivering molecules in vivo and in vitro, especially small molecules, nucleic acids or polypeptides, may be used for the embodiments. Such methods include microspheres, liposomes, other microparticle vehicles or controlled release formulations placed in certain tissues, including blood. Examples of controlled release carriers include semi-permeable polymer matrices in the form of shaped articles, e.g., suppositories, or microcapsules and U.S. Pat. Nos. 5,626,877; 5,891,108; 5,972,027; 6,041,252; 6,071,305, 6,074,673; 6,083,996; 6,086,582; 6,086,912; 6,110,498; 6,126,919; 6,132,765; 6,136,295; 6,142,939; 6,235,312; 6,235,313; 6,245,349; 6,251,079; 6,283,947; 6,283,949; 6,287,792; 6,296,621; 6,309,370; 6,309,375; 6,309,380; 6,309,410; 6,317,629; 6,346,272; 6,350,780; 6,379,382; 6,387,124; 6,387,397 and 6,296,832. Moreover, formulations for administration can include, for example, transdermal patches, ointments, lotions, creams, gels, drops, suppositories, sprays, liquids, and powders.

Cells that may be exposed to, or transfected by, transposons can be obtained from a variety of sources including bacteria, fungi, plants and animals, e.g., a vertebrate or an invertebrate; for example, crustaceans, mollusks, fish, birds, mammals, rodents, ungulates, sheep, swine and humans. Cells that may be exposed to a transposon include, e.g., lymphocytes, hepatocytes, neural cells, muscle cells, a variety of blood cells, stem cells for various tissues and organs and a variety of cells of an organism. These cells include stem cells such as CD34+ hematopoietic stein cells, as well as tissue-specific cell types such as hepatocytes and sinusoidal epithelial cells in liver.

### Transgenic Animals

This document features transgenic non-human animals (e.g., mice, rats, pigs, sheep, goats, or cows). The nucleated cells of the transgenic animals provided herein contain a nucleic acid construct described above. As used herein, “transgenic animal” includes founder transgenic animals as well as progeny of the founders, progeny of the progeny, and so forth, provided that the progeny retain the nucleic acid construct. For example, a transgenic founder animal can be used to breed additional animals that contain the nucleic acid construct.

Tissues obtained from the transgenic animals (e.g., transgenic mice or pigs) and cells derived from the transgenic animals (e.g., transgenic mice or pigs) also are provided herein. As used herein, “derived from” indicates that the cells can be isolated directly from the animal or can be progeny of such cells. For example, brain, lung, liver, pancreas, heart and heart valves, muscle, kidney, thyroid, corneal, skin, blood vessels or other connective tissue can be obtained from a transgenic pig. Blood and hematopoietic cells, Islets of Langerhans, beta cells, brain cells, hepatocytes, kidney cells, and cells from other organs and body fluids, for example, also can be derived from transgenic animals. Organs and cells from transgenic pigs can be transplanted into a human patient. For example, islets from transgenic pigs can be transplanted to human diabetic patients.

Various techniques known in the art can be used to introduce nucleic acid constructs into non-human animals to produce founder lines, in which the nucleic acid construct is integrated into the genome. Such techniques include, without limitation, pronuclear microinjection (U.S. Pat. No. 4,873,191), retrovirus mediated gene transfer into germ lines (Van der Putten et al. (1985) Proc. Natl. Acad. Sci. USA 82, 6148-1652), gene targeting into embryonic stem cells (Thompson et al. (1989) Cell 56, 313-321), electroporation of embryos (Lo (1983) Mol. Cell. Biol. 3, 1803-1814), sperm mediated gene transfer (Lavitrano et al. (2002) Proc. Natl. Acad. Sci. USA 99, 14230-14235; Lavitrano et al. (2006) Reprod. Fert. Develop. 18, 19-23), and in vitro transformation of somatic cells, such as cumulus or mammary cells, or adult, fetal, or embryonic stem cells, followed by nuclear transplantation (Wilmut et al. (1997) Nature 385, 810-813; and Wakayama et al. (1998) Nature 394, 369-374). Pronuclear microinjection, sperm mediated gene transfer, and somatic cell nuclear transfer are particularly useful techniques.

Typically, in pronuclear microinjection, a nucleic acid construct described above is introduced into a fertilized egg; 1 or 2 cell fertilized eggs are used as the pronuclei containing the genetic material from the sperm head and the egg are visible within the protoplasm. Pronuclear staged fertilized eggs can be obtained in vitro or in vivo (i.e., surgically recovered from the oviduct of donor animals). In vitro fertilized eggs can be produced as follows. For example, swine ovaries can be collected at an abattoir, and maintained at 22-28° C. during transport. Ovaries can be washed and isolated for follicular aspiration, and follicles ranging from 4-8 mm can be aspirated into 50 mL conical centrifuge tubes using 18 gauge needles and under vacuum. Follicular fluid and aspirated oocytes can be rinsed through pre-filters with commercial TL-HEPES (Minitube, Verona, Wis.). Oocytes surrounded by a compact cumulus mass can be selected and placed into TCM-199 Oocyte Maturation Medium (Minitube, Verona, Wis.) supplemented with 0.1 mg/mL cysteine, 10 ng/mL epidermal growth factor, 10% porcine follicular fluid, 50 μM 2-mercaptoethanol, 0.5 mg/ml cAMP, 10 IU/mL each of pregnant mare serum gonadotropin (PMSG) and human chorionic gonadotropin (hCG) for approximately 22 hours in humidified air at 38.7° C. and 5% CO₂. Subsequently, the oocytes can be moved to fresh TCM-199 maturation medium which will not contain cAMP, PMSG or hCG and incubated for an additional 22 hours. Matured oocytes can be stripped of their cumulus cells by vortexing in 0.1% hyaluronidase for 1 minute.

Mature oocytes can be fertilized in 500 μl Minitube PorcPro IVF Medium System (Minitube, Verona, Wis.) in Minitube 5-well fertilization dishes. In preparation for in vitro fertilization (IVF), freshly-collected or frozen boar semen can be washed and resuspended in PorcPro IVF Medium to 4×10⁵ sperm. Sperm concentrations can be analyzed by computer assisted semen analysis (SpermVision, Minitube, Verona, Wis.). Final in vitro insemination can be performed in a 10 μl volume at a final concentration of approximately 40 motile sperm/oocyte, depending on boar. Incubate all fertilizing oocytes at 38.7° C. in 5.0% CO₂ atmosphere for 6 hours. Six hours post-insemination, presumptive zygotes can be washed twice in NCSU-23 and moved to 0.5 mL of the same medium. This system can produce 20-30% blastocysts routinely across most boars with a 10-30% polyspermic insemination rate.

Linearized nucleic acid constructs can be injected into one of the pronuclei then the injected eggs can be transferred to a recipient female (e.g., into the oviducts of a recipient female) and allowed to develop in the recipient female to produce the transgenic animals. In particular, in vitro fertilized embryos can be centrifuged at 15,000×g for 5 minutes to sediment lipids allowing visualization of the pronucleus. The embryos can be injected with approximately 5 picoliters of the transposon/transposase cocktail using an Eppendorf Femtojet injector and can be cultured until blastocyst formation (˜144 hours) in NCSU 23 medium (see, e.g., WO/2006/036975). Rates of embryo cleavage and blastocyst formation and quality can be recorded.

Embryos can be surgically transferred into uteri of asynchronous recipients. For surgical embryo transfer, anesthesia can be induced with a combination of the following: ketamine (2 mg/kg); tiletamine/zolazepam (0.25 mg/kg); xylazine (1 mg/kg); and atropine (0.03 mg/kg) (all from Columbus Serum). While in dorsal recumbency, the recipients can be aseptically prepared for surgery and a caudal ventral incision can be made to expose and examine the reproductive tract. Typically, 100-200 (e.g., 150-200) embryos can be deposited into the ampulla-isthmus junction of the oviduct using a 5.5-inch TOMCAT® catheter. After surgery, real-time ultrasound examination of pregnancy can be performed using an ALOKA 900 ultrasound scanner (Aloka Co. Ltd, Wallingford, Conn.) with an attached 3.5 MHz trans-abdominal probe. Monitoring for pregnancy initiation can begin at 23 days post fusion and can be repeated weekly during pregnancy. Recipient husbandry can be maintained as normal gestating sows.

In somatic cell nuclear transfer, a transgenic animal cell (e.g., a transgenic pig cell) such as an embryonic blastomere, fetal fibroblast, adult ear fibroblast, or granulosa cell that includes a nucleic acid construct described above, can be introduced into an enucleated oocyte to establish a combined cell. Oocytes can be enucleated by partial zona dissection near the polar body and then pressing out cytoplasm at the dissection area. Typically, an injection pipette with a sharp beveled tip is used to inject the transgenic cell into an enucleated oocyte arrested at meiosis 2. In some conventions, oocytes arrested at meiosis 2 are termed “eggs.” After producing a porcine embryo (e.g., by fusing and activating the oocyte), the porcine embryo is transferred to the oviducts of a recipient female, about 20 to 24 hours after activation. See, for example, Cibelli et al. (1998) Science 280, 1256-1258 and U.S. Pat. No. 6,548,741. For pigs, recipient females can be checked for pregnancy approximately 20-21 days after transfer of the embryos.

Standard breeding techniques can be used to create animals that are homozygous for the target nucleic acid from the initial heterozygous founder animals. Homozygosity may not be required, however. Transgenic animals described herein can be bred with other animals of interest.

In some embodiments, a nucleic acid of interest and a selectable marker can be provided on separate transposons and provided to either embryos or cells in unequal amount, where the amount of transposon containing the selectable marker far exceeds (5-10 fold excess) the transposon containing the nucleic acid of interest. Transgenic cells or animals expressing the nucleic acid of interest can be isolated based on presence and expression of the selectable marker. Because the transposons will integrate into the genome in a precise and unlinked way (independent transposition events), the nucleic acid of interest and the selectable marker are not genetically linked and can easily be separated by genetic segregation through standard breeding. Thus, transgenic animals can be produced that are not constrained to retain selectable markers in subsequent generations, an issue of some concern from a public safety perspective.

Once transgenic animal have been generated, expression of a target nucleic acid can be assessed using standard techniques. Initial screening can be accomplished by Southern blot analysis to determine whether or not integration of the construct has taken place. For a description of Southern analysis, see sections 9.37-9.52 of Sambrook et al., 1989, Molecular Cloning, A Laboratory Manual, second edition, Cold Spring Harbor Press, Plainview; NY. Polymerase chain reaction (PCR) techniques also can be used in the initial screening. PCR refers to a procedure or technique in which target nucleic acids are amplified. Generally, sequence information from the ends of the region of interest or beyond is employed to design oligonucleotide primers that are identical or similar in sequence to opposite strands of the template to be amplified. PCR can be used to amplify specific sequences from DNA as well as RNA, including sequences from total genomic DNA or total cellular RNA. Primers typically are 14 to 40 nucleotides in length, but can range from 10 nucleotides to hundreds of nucleotides in length. PCR is described in, for example PCR Primer: A Laboratory Manual, ed. Dieffenbach and Dveksler, Cold Spring Harbor Laboratory Press, 1995. Nucleic acids also can be amplified by ligase chain reaction, strand displacement amplification, self-sustained sequence replication, or nucleic acid sequence-based amplified. See, for example, Lewis (1992) Genetic Engineering News 12,1; Guatelli et al. (1990) Proc. Natl. Acad. Sci. USA 87, 1874-1878; and Weiss (1991) Science 254, 1292-1293. At the blastocyst stage, embryos can be individually processed for analysis by PCR, Southern hybridization and splinkerette PCR (see, e.g., Dupuy et al. Proc Natl Acad Sci USA (2002) 99(7):4495-4499).

Expression of a nucleic acid sequence encoding a polypeptide in the tissues of transgenic animals can be assessed using techniques that include, without limitation, Northern blot analysis of tissue samples obtained from the animal, in situ hybridization analysis, Western analysis, immunoassays such as enzyme-linked immunosorbent assays, and reverse-transcriptase PCR (RT-PCR).

### Articles of Manufacture

Isolated nucleic acids and polypeptides described herein can be combined with packaging material and sold as a kit, e.g., for introducing DNA into a host cell. Components and methods for producing articles of manufactures are well known.

Articles of manufacture also may include reagents for carrying out the methods disclosed herein (e.g., a buffer or control nucleic acids). Instructions describing how the nucleic acids and polypeptides can be used for introducing DNA into a host cell also may be included in such kits.

The invention will be further described in the following examples, which do not limit the scope of the invention described herein.

### EXAMPLES

### Example 1

### Methods and Materials

pPTn1-SE—Using T3-rev [TCTCCCTTTAGTGAGGGTTAATT] (SEQ ID NO: 38) and T7-rev [TCTCCCTATAGTGAGTCGTATTA] (SEQ ID NO: 39) primers, a 102 bp PCR product of pKT2-SE that provides T7 and T3 polymerase binding sites oriented towards the inverted repeats of the PTn transposon and separated by a short multiple cloning site was cloned into the MscI site of prePTn1(−1). prePPTn1(−1) was made by cloning a 0.65 kb BamHI to KpnI fragment of pCR4-PPTN1A into pK-A3 opened from KpnI to BamHI. pCR4-PPTN1A was created by topo cloning a 0.65 kb PCR product amplified from prePPTN1(−2) using oligos PPTN-F1 (BamHI) [AAGGATCCGATTACAGTGCCTTGCATAAGTAT] (SEQ ID NO: 40) and PPTN-R2 (KpnI) [AAGGTACCGATTACAGTGCCTTGCATAAGTATTC] (SEQ ID NO: 41) into pCR4—Topo (Invitrogen). prePPTN1(−2) was created by amplifying the majority of pBluKS-PPTN5 (Leaver, Gene 2001, 271(2):203-214) with oligos PPTN-OL1 [CCAGTTTGTTCAGTAATGATCTCCAAC] (SEQ ID NO: 42) and PPTN-OR1 [CCAGGTTCTACCAAGTATTGACACA] (SEQ ID NO: 43). The PCR fragment was then self-ligated to produce an empty transposon with a single MscI site in its interior.

pPTn2-SE—PTn2-SE was created in an identical manner as pPTn1-SE except that the creation of prePPTN2(−2) utilized oligo PPTN-OL2 [CCATCTTTGTTAGGGGTTTCACAGTA] (SEQ ID NO: 44) with PPTN-OR1, which essentially removed an additional 147 bp of conserved 5′UTR sequence from within the ITRs as compared to prePPTN1(−2).

pPTn1P-GeN/pPTn2P-GeN—pPTn1P-GeN and pPTn2P-GeN were produced by cloning a 3.4 kb XmaI to NheI fragment of pKT2P-GeN (Clark et al. BMC biotechnology 2007, 7:42), which contained the human PGK promoter and mini-intron, EGFP, the encephalomyocarditis virus internal ribosome site, neomycin phosphotransferase, and the rabbit beta-globin poly(A) signal, into either pPTn1-SE or pPTn2-SE, respectively.

pKUb-PTs1/pKUb-PTs2-pKUb-PTs* was made by replacing the SB11 gene in pKUb-SB11 with PTs1 or PTs2 by cloning a 1.0 kb BamHI to NheI fragment from pCR4-PPTs1 or pCR4-PPTs2 into pKUb-SB11 from NheI to BamHI. pCR4-PPTs1 was made by cloning a PCR fragment of pBluKS-PPTN4 (Leaver, supra) amplified with primers CDS-PPTs-F1 [AAAGCTAGCATGAAGACCAAGGAGCTCACC] (SEQ ID NO: 45) and CDS-PPTs-R1 [AAGGATCCTCAATACTTGGTAGAACC] (SEQ ID NO: 46) into pCR4-Topo (Invitrogen). pCR4-PPTs2 was made by a nearly identical amplification using CDS-PPTs-F1 alt [AAAGCTAGCATGGGAAAGACCAAGGAGCTCACC] (SEQ ID NO: 47) and CDS-PPTs-R1.

pKC-PTs1/pKC-PTs2—The PTs coding regions were placed behind the mCAGs promoter by cloning a 1.0 kb NheI to EcoRI fragment of pKUb-PTs 1 and pKUb-PTs2 containing the PPTN transposase (PTs1 and PTs2, respectively) into pK-mCAG opened from EcoRI to NheI. pK-mCAG was made by cloning the mCAG promoter from pSBT-mCAG (Ohlfest et al. Blood 2005, 105(7):2691-2698) as a 0.96 kb SmaI to EcoRI (filled) fragment into pK-SV40(A)ₓ₂ opened with AflII (filled).

pKUb-SB11—The construction of pKUb-SB11 has been described by Clark et al., supra.

pKC-SB11—pKC-SB11 was made by cloning a 1.05 kb NheI to EcoRI fragment from pKUb-SB11 into pK-mCAG (see Clark et al., supra) opened from EcoRI to NheI.

pCMV-β is available from Clontech (Mountainview, Calif.).

pPTnP-PTK—A 2.7 kb PvuII to PvuII fragment of pKP-PTK TS (Clark et al., supra) was cloned into the EcoRV site of pPTn2-RV to make pPTnP-PTK. pPTn2-RV was made by cloning KJC-Adapter 4 [TCTCCCTTTAGTGAGGGTTAATTGATATCTAATACGACTCACTATAGGGAGA] (SEQ ID NO: 48) into the MscI site of prePTn2(−1) creating T7 and T3 polymerase binding sites orientated out towards the inverted repeats of the PTn transposon and separated by an EcoRV site.

Cell culture and transposition assays. HT1080, HeLa, CHO-K1, NIH-3T3, and Vero cells are available from ATCC. TT and DF1 cells were kind gift from the laboratory Dr. Douglas Foster, University of Minnesota (Schaefer-Klein et al. Virology 1998, 248(2):305-311; Kong et al Virus research 2007, 127(1):106-115). The isolation of PEGE cells has been described by Clark et al., supra. CHO-K1 cells were grown in DMEM-F12 while all other cell lines were cultured with DMEM. Both mediums were enriched with 10% FBS, 1× Penn/Strep, and 1×L-Glutamine. PEGE cells were also enriched with insulin at 10 ug/mL

Transposition assays were carried out after seeding cells in six well plates to achieve 60-80% confluency prior to transfection with DNA complexed with TranslT-LT1 transfection reagent (Minis Bio Corporation, WI). Transfections were carried out according to manufacturers instructions with a ratio of 3:1 lipid:DNA. Two days after transfection, cells were isolated from their wells with trypsin and collected by centrifugation. Two replicates of 30,000 cells were plated on 100 mm dishes and selected in the appropriate selectable media. HT1080 cells were selected in 600 ug/ml of G418. For puromycin selection, HT1080, HeLa, Cho-K1, NIH-3T3, Vero, TT1, DF1, and PEGE cells were selected under 0.65, 0.4, 8.0, 1.5, 1.8, 0.35, 0.8, and 0.3 ug/mL puromycin, respectively. After colony formation, typically 9-12 days under selection, colonies were stained with methylene blue and counted.

Southern hybridization. Genomic DNA from independent clones derived after transfection with Passport transposons (pPTn2P-PTK) and Passport transposase (pKC-PTs1) was isolated using standard methods. Approximately 10 ug of DNA was digested with AseI and run on a 0.7% agarose gel. The DNA was transferred to a positively charged nylon membrane using 10×SSC and standard methods. The membrane was hybridized with a random primed fragment of pKP-PTK-TS isolated after digestion with XmaI. This probe contains the bulk of the puromycin-thymidine kinase gene, about 1.5 kb.

Cloning junction fragments. Blocked linker-mediated PCR was performed as described by Clark et al., supra, except that DNA was obtained from colonies of cells that had been dried and stained with methylene blue. Briefly, genomic DNA was digested with a cocktail of restriction enzymes, including XbaI, NheI, AvrII, and SpeI. The DNA was ligated to a blocked linker made by annealing the oligos primerette-long [CCTCCACTACGACTCACTGAAGGGCAAGCAGTCCT (SEQ ID NO: 49) AACAACCATG] (SEQ ID NO: 50) and blink-XbaI [5 P-CTAGCATGGTTGTTAGGACTGCTTGC-3′P]. Nested PCR was performed on the ligated DNA to specifically amplify junctions between the Passport transposon and genomic DNA. The transposon-specific primers for the primary PCR included PTn-IRDR(L)-O1 [GTGTTGGTCCATTACATAAACTCACGATGAA] (SEQ ID NO: 51) or PTn-IRDR(R)-O1 [GGGTGAATACTTATGCACCCAACAGATG] (SEQ ID NO: 52), transposon-specific primers for the secondary PCR reactions included PTn-IRDR(L)-O2 [GCATGACAAAATGTAGAAAAGTCCAAAGG] (SEQ ID NO: 53) and PTn-IRDR(R)-O2 [CAGTACATAATGGGAAAAAGTCCAAGGG] (SEQ ID NO: 54).

Phylogenetic Analysis. The 1626 by DNA sequence of PPTN (Passport) was used to query the entire ENSEMBL (www.ensembl.org) genome database using BLASTN. Consensus DNA sequences were derived, as described by Leaver, supra, from a minimum of seven of the most similar sequences from each genome. Deduced consensus transposase amino acid sequences were aligned using ClustalW and phylogenetic trees generated as described by Leaver, supra. The Atlantic salmon (Salmo salar) and rainbow trout (Oncorhyncus mykiss) EST and tentative consensus cDNA databases (see http// site at compbio.dfci.harvard.edu/tgi/) were also interrogated with PPTN using BLASTN and sequences assembled into consensus polypeptides as described for genome sequences.

### Example 2

### Native Passport is Competent for Transposition

To test the cis and trans acting components of the Passport transposon system, the transposase gene was separated from the transposon inverted terminal repeats (ITRs). Comparison of the ITRs of Passport with those of related Tel family members revealed that a cis element between the ITR and the transposase coding region that contains mostly 5′-untranslated region (5′UTR) seemed to be conserved to a similar degree as the transposase coding region. To examine the importance of this conserved region of the Passport transposon, we prepared two transposon vectors, one that maintained (pPTn1) and one that eliminated (pPTn2) this sequence (FIG. 1A). The wild-type Passport transposase (PTs1) open reading frame is 339 amino acids, whereas the coding region of SB and Frog Prince are 340 amino acids long, differing in the presence of an additional amino acid in the penultimate position at their N-termini.

To examine whether or not this additional amino acid could influence transpositional activity, a second transposase (PTs2) was made that added a glycine residue at the penultimate position (FIG. 1B). Both PTs1 and PTs2 coding sequence were cloned behind the mCAG promoter or Ubiquitin promoter, yielding four transposase expression vectors-pKC-PTs1, pKC-PTs2, pKUb-PTs1, and pKUb-PTs2 (FIG. 1C).

To test the Passport transposon system in human cells, a dicistronic expression cassette consisting of the human PGK promoter driving expression of green fluorescent protein (GFP) and neomycin phosphotransferase (neo^{R}) was cloned between the ITRs of both pPTnl-SE and pPTn2-SE to produce pPTn1P-GeN and pPTn2P-GeN, respectively. The resultant transposons were transfected into HT1080 cells, a human fibrosarcoma cell line, with an equimolar source of transposase expression vector (pKC-PTs1 or pKC-PTs2) or with a non-transposase control DNA that instead expresses β-galactosidase (Bgal). Following transfection, replicates of 30,000 cells were plated and selected in G418 for 10-14 days, fixed, stained and enumerated. Cells that integrated the neo^{R} cargo of the transposon into their genomes were able to withstand selection in G418 and gave rise to colonies of cells. In all cases, when a Passport transposon was paired with a source of transposase, there was a significant increase in the number of G418 resistant colonies compared to transfection with the βgal expressing vector (FIG. 2).

Native and N-terminally modified Passport transposases (PTs1 or PTs2) both enhanced colony formation in our assays, suggesting the native Passport transposase is functional, and that conservation of the penultimate N-terminal length of Tc1 transposases is not strictly required for activity. The provision of native transposase (PTs1) with native transposon sequences (PTn1) resulted in more than a 10-fold increase in colony formation compared to the no-transposase control (Bgal). Although pairing of the native transposase (PTs1) with PTn2, which lacks the 147 by 5′-UTR, resulted in an increase in colony formation when compared to background (Bgal), the number of resistant colonies generated was significantly reduced in comparison to pairing with PTn1 that contains the 147 bp cis element. However, the difference in transpositional activity for PTn1P and PTn2P was not statistically significant when coupled with PTs2, in which a consensus N-terminal length change was made.

### Example 3

### Passport is Sensitive to Overproduction Inhibition

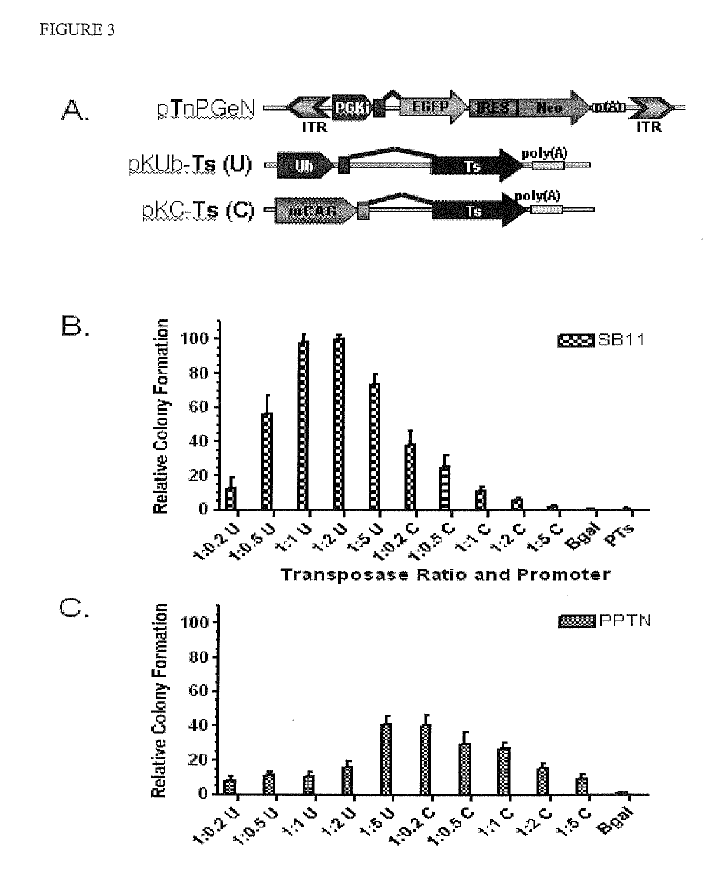

Overproduction inhibition, in which excessive wild-type transposase reduces the rate of excision of a target element, is a hallmark of Tc1/mariner elements (Hartl et al., supra) and an important mechanism for titrating/inhibiting in vivo transposition. We thus undertook an analysis of this effect for Passport and compared its sensitivity to that of the well-characterized SB transposon system (Geurts et al. Mol Ther 2003, 8(1):108-117). A series of transfections were performed with varying ratios of transposase to transposon vector in order to measure the effect of increasing transposase concentration on the rate of transposition. In addition, two promoters were used to drive expression of the Passport transposase, to span a broad range of transposase expression levels (FIG. 3) human Ubiquitin C and mCAG, a shortened version of the hybrid of the cytomegalovirus early enhancer and the chicken beta-actin promoter. In HT1080 cells, the expression of a reporter gene from the mCAGs promoter is between 5 and 10-fold higher than from the Ubiquitin promoter (data not shown). To provide a range of transposase expression, a constant amount of transposon (pPTnP-GeN, 75 femtomoles) was co-transfected with transposase vector containing either the Ubiquitin or mCAGs promoter (pKT C-PTs 1 or pKUb-PTs1) at a Tn:Ts molar ratio of 1:0.2, 1:0.5, 1:1, 1:2, or 1:5 (corresponding to 15, 37.5, 75, 150, and 375 femtomoles of transposase plasmid). The total amount of transfected DNA was kept at 2 μg by supplementing with pCMV-βgal DNA. To compare the response with the SB transposon system, analogous reactions were performed with an SB transposon (pKT2P-GeN) and SB11 transposase expressed from Ubiquitin and mCAGs promoters (pKUb-SB11 and pKC-SB11). Following transfection, two replicates of 30,000 cells were plated and selected in G418 for 10-14 days, fixed, stained and enumerated. Our previous studies indicated that a molar ratio of 1:1 SB transposon to SB transposase expressed from the human Ubiquitin C promoter resulted in near optimal transposition rates for the SB transposon system. Therefore to correct for any variation in transfection or selection, a 1:1 ratio of pKT2P-GeN:pKUb-SB11 was included as in internal standard for every transfection. The relative sensitivity of the two transposon systems to overproduction inhibition is presented in FIGS. 3C & D, where colony formation is expressed relative to the contemporary pKT2P-GeN:pKUb-SB11 internal standard. As shown in FIG. 3C, the hyperactive SB system resulted in the generation of significantly more colonies than the native Passport system (FIG. 3D) at their respective optimal Tn:Ts ratios. As expected, the SB transposon system is sensitive to overproduction inhibition, with a suppression of transposition at transposase expression levels exceeding that provided by optimal conditions. The peak transpositional activity for Passport was observed using a 1:5 ratio of pPTnP-GeN:pKUb-PTs1 or a 1:0.2 ratio of pPTnP-GeN:pKC-PTs1, beyond which increasing transposase expression resulted in reduced transposition, indicating that Passport is indeed susceptible to overproduction inhibition. Interestingly, despite using identical promoters in the SB and Passport transposase expression constructs, optimal transposition and the emergence of overproduction inhibition for Passport occurred under conditions expected to correspond to significantly higher levels of transposase expression. We can estimate that optimal transposition for Passport requires more than double the amount of transposase expression as SB, since their maximal transposition occurred at Tn:Ts molar equivalents of 1:5 and 1:2, respectively. This could result from differences in the translational efficiency or stability of the encoded transposases. More likely, this result could derive from differences in the affinities of the transposases for their corresponding transposon, or from innate variance in transpositional activity, disparities not unexpected when comparing native and hyperactive transposon systems.

### Example 4

### Passport is Active in Cells of Diverse Vertebrate Origin

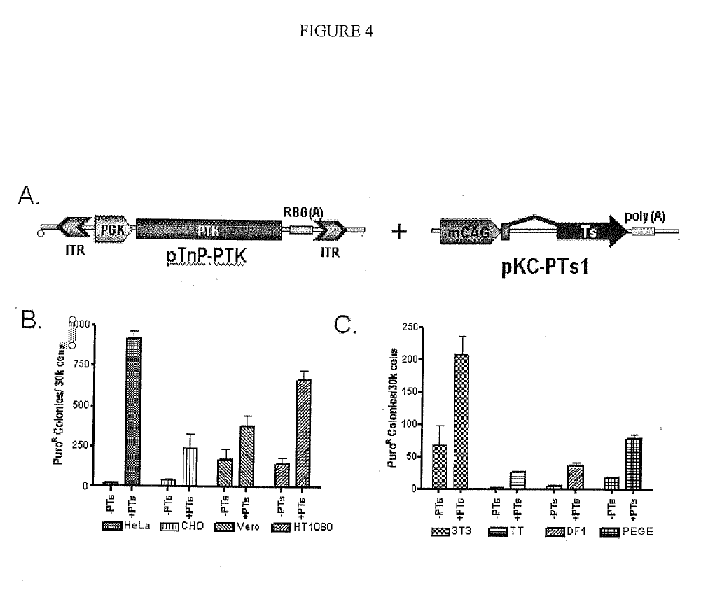

The SB, Frog Prince, and himar1 transposon systems are active in a wide array of vertebrate cells, although to differing degrees. To assess the ubiquity of Passport function, we undertook an analysis of TnT in human (HeLa, HT1080), monkey (Vero), pig (PEGE), hamster (CHO), mouse (3T3), chicken (DF1) and turkey (TT) cells. For this experiment, we constructed a Passport transposon containing a puromycin thymidine kinase fusion protein under the direction of the mouse PGK promoter (pPTn2P-PTK). Cells were transfected with the pPTn2P-PTK transposon along with a Passport transposase expression construct (pKC-PTs1) at a Tn:Ts molar ratio of 1:0.5, or with the molar equivalent of pCMV-βgal, as a transposase negative control. See bottom panel of FIG. 4.

Following transfection, replicates of 30,000 cells were plated and selected in puromycin, fixed, stained and enumerated. In all cases, Passport-dependent TnT resulted in the generation of a number of puromycin resistant colonies significantly exceeding that observed for controls lacking Passport expression, in the case of HT1080 cells reflecting up to a 20-fold enhancement (FIG. 4). Transpositional enhancement varied between cell types (as did background resistant colony formation), although comparing relative transpositional activity across cell lines may be confounded by the fact that transfections were conducted under identical conditions that may be suboptimal for some cell lines. Nonetheless, native Passport is functional in cells from a broad sampling of vertebrate species.

### Example 5

### Molecular Characterization of Passport Transposition

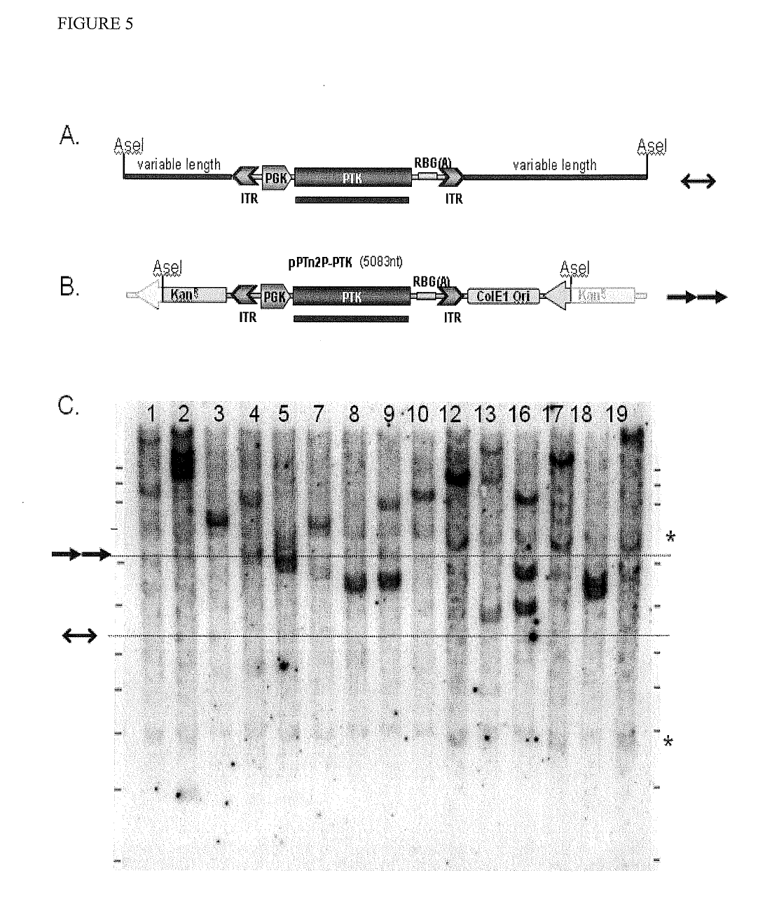

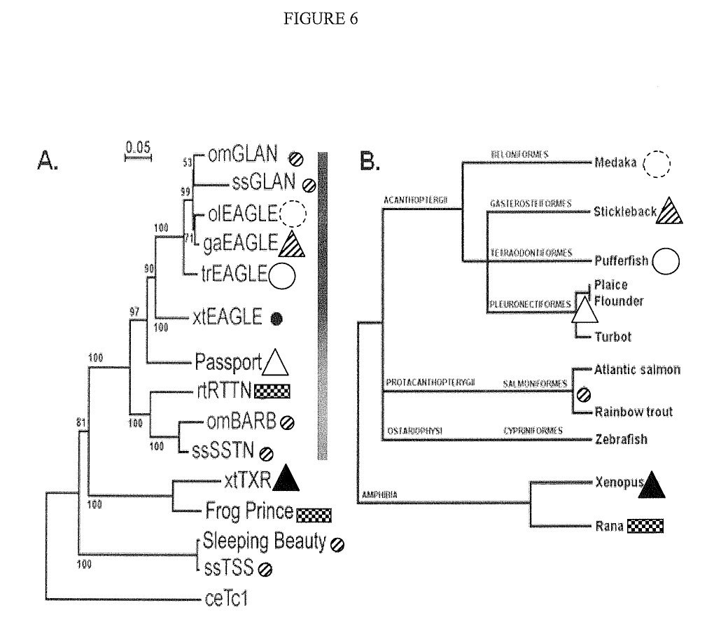

Although the enhanced generation of resistant colonies in the presence of transposase suggests TnT, it does not prove it. We therefore undertook the validation of transposition by molecular analysis. In addition, we sought to examine the number of transposition events per cellular clone, and to define the preferred integration site for the Passport transposon system. For each clone, transposition is supported by hybridizing fragments of varying length, corresponding to genomic restriction sites at varying distances from the transposon insertion (FIG. 5A). Unlike transposition, transgenesis by unfacilitated DNA integration most often results in the formation of multi-copy concatemers that are expected to result in a predictable restriction enzyme fragment derived from sites within the transposon vector (FIG. 5B). The Southern analysis of DNA isolated from fifteen HT1080 clones revealed that Passport indeed had transposed the PTK-selection cassette from the pPTn2P-PTK transposon into the human genome, with 1 to 4 integrations per cellular clone (FIG. 5C). With these transfection techniques, an average of about 2-3 precise transposition events per clone are expected based on the Southern analysis of these 15 clones. In contrast to the apparent transposition events, only clone 5 contains a hybridizing band near the predicted size of a concatemer; in addition to this potential concatemer band, clone 5 has additional bands that likely represent transposition events.

To further verify TnT by Passport, and to characterize the insertion target sites and preferences within HT1080 cells, junction fragments between the transposon and host genome were cloned and sequenced. Passport, like other Tc1 transposons, is expected to integrate into a TA dinucleotide and cause target-site duplication of the TA sequence at the ITR boundary. Table 1 lists 27 independent insertion events identified in HT1080 cells. In Table 1, the integration sites show what is outside the left ITR (L), the TA that is duplicated upon integration, and the sequence outside the right ITR (R). Table 1 includes SEQ ID NOs: 55-110. The first sequence indicates the sequence found in the donor plasmid (shaded), while the remaining represent 27 Passport integrations sites all of which occurred by TnT as indicated by the exact junction at the ITR with a TA dinucleotide from the genome. In each case the sequence represented in CAPS was cloned by blocked LM-PCR and the sequence in lower case was derived from genome sequence data. In many cases, the Passport transposon integrated into known or (predicted) genes (Locus). The transposon integrations targeted a wide variety of chromosomal positions (Chrm Pos). Using ProTIS [30], we calculated the Vstep associated with each integration site. The Vstep values are as defined by Geurts et al.

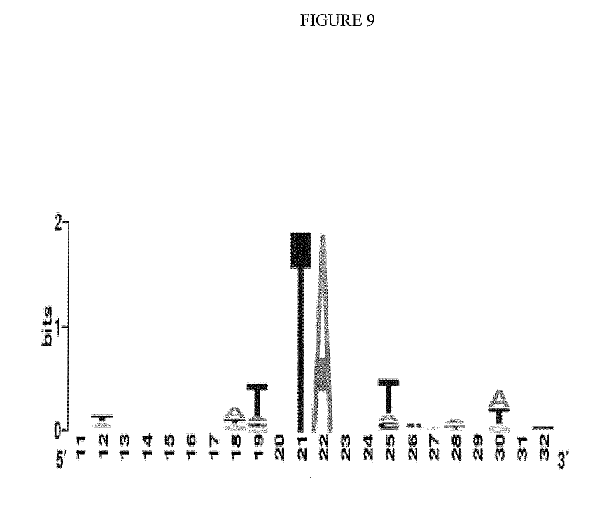

All of these events in Table 1 demonstrate integration of the transposon into a TA within the human genome, validating genuine transposition. Comparison of the cloned junction sequences to the human genome by Blast analysis was undertaken to define the locations of transposon insertions at a genomic level. This analysis revealed that insertions were randomly dispersed across the human genome (Table 1—Chrm Pos). However, insertions were found in genes in 63% of the cloned junctions. Although only 27 junctions were cloned and sequenced, integration site sequences were compared to each other to characterize any preference Passport might have for sequence composition beyond the absolute requirement for a TA at the integration site. Like SB (Vigdal, J Mol Biol. 2002 323 (3):441-52), some minor preferences are apparent and may differ from those of SB and other Tel elements (FIG. 9). Since the target-site preference of SB may depend more on local DNA deformity than primary sequence, we calculated the Vₛₜₑₚ, a measure of local DNA deformity, at each target site using ProTIS. Assuming a similar representation of Vₛₜₑₚ patterns across the human genome as found within 3.2 Mbp of mouse chromosome 1 (Table 1 Geurts et al., 2006) Passport integrated into semi-preferred and preferred sites 2.9× and 3.9× more often than basal TA sites. Although there is a preference for these sites, the Vₛₜₑₚ seems to have less of an impact for Passport integrations as compared to SB (Geurts et al. Nucleic Acids Res 2006, 34(9):2803-2811).

### Example 6

### Passport-Like Transposons are Present in Other Fish and Amphibian Genomes

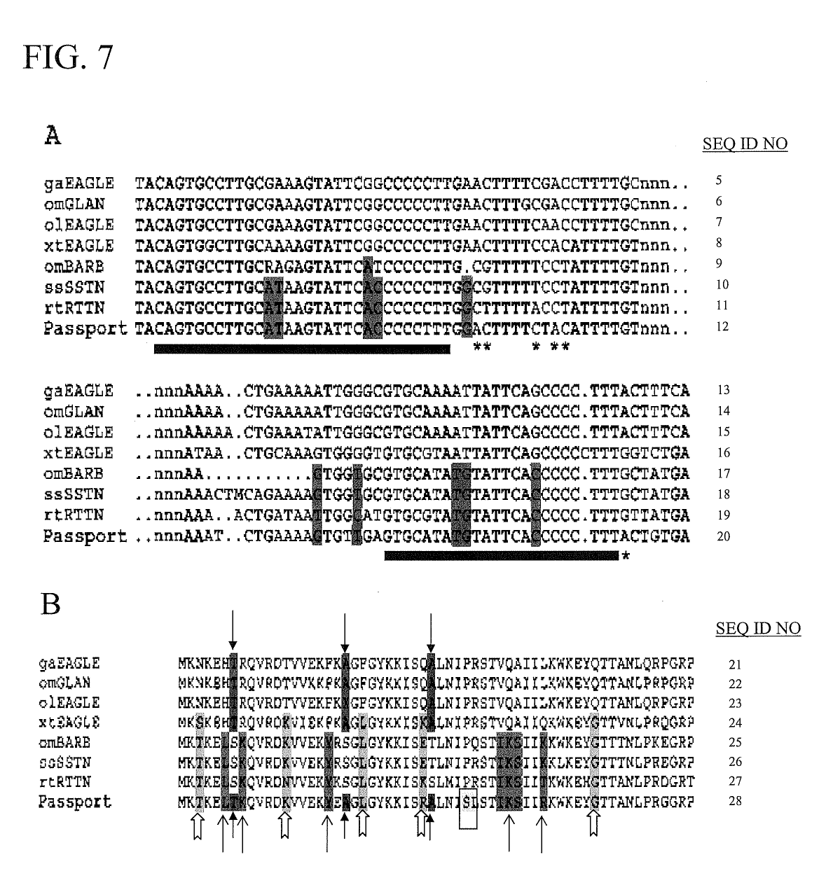

The availability of sequenced genomes provides an opportunity to compare and categorize all transposons within a species and derive representative consensus sequences with a minimum of experimental bias. Passport elements originally isolated from plaice have been identified in other flatfish, including flounder and turbot (99% and 98% DNA identity over the entire 1.6 kb element)—suggesting a recent horizontal transfer of Passport or exceptional conservation of these sequences. A recent search of the ENSEMBL genome database revealed the presence of related transposons with high nucleotide identity (>80%) to Passport transposase in the genomes and EST collections of the amphibian Xenopus tropicalis, and the fish species pufferfish (Takafugu rubripes), stickleback (Gasterostreus aculeatus), medaka (Oryzis latipes), Atlantic salmon (Salmo salmar) and rainbow trout (Oncorhynchus mykiss). Passport-like transposons were absent from all other ENSEMBL genomes, including those of the zebrafish (Danio rerio), despite the wide range and high copy number of other Tc1-like elements in this species. Comparison of the encoded transposase amino acid sequences show that relatives of Passport form a distinct family of Tc1-like transposons that is further divided into two subfamilies, including Eagle/Glan and SSTN/Barb (FIG. 7). The salmonids (salmon and rainbow trout) contain members of both subfamilies, whilst X. tropicalis, pufferfish, stickleback and medaka contain only the Eagle/Glan subfamily. The structure of Passport is somewhat intermediate between that of Eagle/Glan and SSTN/Barb, in that its terminal inverted repeats bear a strong resemblance to SSTN/Barb (FIG. 8A) whereas its transposase coding region seems to bear more resemblance to the Eagle/Glan subfamily than other members of the SSTN/Barb subfamily. Importantly, alignment of the DNA-binding domains of the transposases demonstrates a distinction between Eagle/Glan and Passport/SSTN/Barb (FIG. 8B), a difference that may functionally be connected to differences that are also present in the inverted terminal repeats of these elements.

In summary, Passport is a naturally occurring, active vertebrate Tel transposon. Passport supports impressive rates of transposition, achieving levels up-to half that for observed for SB11, itself a hyperactive mutant that is about 3-fold more active than the originally reanimated SB10 (Geurts et al. Mol Ther 2003, 8(1):108-117). The identification of a natural and functional vertebrate Tel-like transposon may provide unique insights into the mechanisms and regulation of transposition in vertebrates. Efforts to develop hyperactive transposases for application to TnT and gene therapy have applied both structure-based and phylogenetics-informed approaches. Indeed, the native Passport transposase sequence has been considered in phylogenetic-based improvements to SB and it contains several residues that have been synthetically introduced to generate hyperactive SB mutants, including; L205 & VR207/8 [Baus et al. Mol Ther 2005, 12(6):1148-1156.], R130 & Q243 [Geurts et al., 2003, supra]. Changes have also been made in the cis-acting ITR [Zayed et al. Mol Ther 2004, 9(2):292-304; Cui et al. J. Mol. Biol. 2002, 318(5):1221-1235.], as well as the spacer sequence between the ITRs of the SB transposon [Izsvak et al. J. Mol. Biol. 2000, 302(1):93-102, Zayad et al. supra], resulting in the development of improved transposons, and evidence that only flanking IR/DR are required to constitute an effective transposon.

In this study, we examined the effect of inclusion of the 147 base-pair cis-element (5′UTR) within Passport transposons as well as adding a glycine residue as the second amino acid to the Passport transposase. The inclusion of the 5′UTR cis-element resulted in twice as much transposition as when it was excluded. Additionally, although the effect on transposition was apparent when either native (PTs1) or N-terminal expanded (PTs2) transposase was used, a more dramatic (and statistically significant) effect was revealed for the native transposase. This suggests that the 147 by 5′-UTR is a cis-acting sequence that is functionally tuned in some way to native transposase. The location of this cis-element mirrors that of a similarly positioned element in the wild-type SB transposon that together with an element within the right IR/DR directs convergent inward-directed transcription. Transcription from the SB 5′-UTR was found to be stimulated by the host-encoded high-mobility group 2-like 1 (HMG2L1) protein, which was also found to bind to it. In contrast to our observation that native Passport transposase enhances transposition when combined with the 5′-spacer, SB transposase binds to the HMG2L1 protein and antagonizes transcription. Although yet to be explored by biochemical techniques, these differing characteristics suggest that the observed functional interaction between the N-terminus of the native Passport transposase and the 5′-spacer is distinct from that currently described for SB.

An examination of genome sequence data for diverse organisms shows that Tel elements related to Passport are also present in X. tropicalis and in other fish species. In X. tropicalis these transposons have been termed Eagle [Sinzelle et al. Gene 2005, 349:187-196.] and in rainbow trout Glan and Barb [Krasnov et al. BMC Genomics 2005, 6:107.]. Our recent database analysis indicates that indeed there may be several intact Eagle elements in X tropicalis. Since both Eagle/Glan and Barb/SSTN/RTTN transposons exist in salmonid genomes it is likely that they represent two distinct transposons rather than members of a common heterogeneous transposon population.

Mobile genetic elements are theoretically capable of transferring horizontally between genomes as well as the more likely scenario of being inherited vertically. Members of the Eagle/Glan family are phylogenetically widespread and their distribution is generally in agreement with the accepted phylogeny for these species. The most parsimonious model for their presence in a broad range of genomes is one of vertical transmission and occasional loss, for example from the zebrafish line. Unlike the widespread nature of the Eagle/Glan subfamily, in fish species SSTN/Barb appears to be restricted to salmonids based on the currently available sequence data. In addition, closely related transposons have been found in frogs (Rana, RTTN, Leaver 2001). Therefore a vertical model for transmission of this family would require loss from numerous species of fish, and from at least one amphibian line leading to X. tropicalis. Thus a horizontal model of transmission provides a more parsimonious explanation of the distribution of SSTN/Barb/RTTN transposons. Passport transposons, appear to be an intermediate between the Eagle/Glan group and the SSTN/Barb group of transposons. It is therefore also possible that a form of transposon “hybridization” has resulted in the creation of Passport as a function of recent transposon activity within Pleuronectid genomes.

Passport seems to have a highly restricted phylogenetic distribution, so far found only in pleuronectid flatfish genomes (plaice, flounder and turbot). These flatfish are estimated to have shared a common ancestor only 6 million years ago and consequently Passport representatives from these species share >97% nucleotide identity. It is an intriguing to consider whether Passport invaded the genome of Pleuronectiformes following their colonization of a new habitat after the evolutionary emergence of “flatfish”. Or, alternatively Passport may have arisen in (or invaded) a morphologically “normal” ancestor to Pleuronectids, which raises the question of whether transposon activity contributed to the genomic innovation required for the evolution of “flatness”. With advances in rapid high-throughput sequencing, genome sampling of flatfish should provide data on not only more exhaustive sequence of Passport transposons, but also provide the genomic context of these insertions. The degree of conservation/difference in the locations of transposon loci could provide insight into the history of Passport activity.

### OTHER EMBODIMENTS

It is to be understood that while the invention has been described in conjunction with the detailed description thereof, the foregoing description is intended to illustrate and not limit the scope of the invention, which is defined by the scope of the appended claims.

Accordingly, embodiments include an isolated transposase comprising: a polypeptide that has an amino acid sequence that has at least 80% identity to SEQ ID NO: 29, with the polypeptide specifically binding to the a nucleic acid fragment that comprises an inverted terminal repeat sequence of at least one of SEQ ID NO:34 and SEQ ID NO:35, with the polypeptide catalyzing the integration of a target nucleic acid into a vertebrate cell. The target nucleic acid may be integrated into the genome of the vertebrate cell. The transposase may comprise SEQ ID NO:29. A nucleic acid may encoding the transposase. The nucleic acid may be a portion of a plasmid. Cells may be generated that comprise the transposon and/or transposase as described herein.

Embodiments include a composition comprising an isolated transposon and/or transposase disposed in a carrier as described herein. Such a composition may be administered as described herein. Cells as described herein may be treated with the transposons and/or transposases.

Embodiments include a transposon comprising a first nucleic acid fragment with at least 80% identity to SEQ ID NO: 34 and a second nucleic acid fragment with at least 80% identity to SEQ ID NO: 35, wherein the transposon specifically binds to a polypeptide having the sequence of SEQ ID NO:29. The transposon may be further comprising a target nucleic acid fragment that is located between the first nucleic acid fragment and the second nucleic acid fragment, wherein the target nucleic acid is mobilizable by the polypeptide to be integrated into the genome of a vertebrate cell.

Embodiments include a gene transfer system to introduce DNA into the DNA of a cell comprising: a transposase or a nucleic acid encoding a transposase, with the transposase having a sequence with at least 80% identity to SEQ ID NO:29; and a transposon that comprises a target nucleic acid that is specifically bound by (and mobilizable by) the transposase into a genome of a vertebrate cell. The transposon may comprise, e.g., SEQ D NO:34 and/or SEQ ID NO:35. The target nucleic acid may further comprise a promoter. The transposase may be provided as RNA, as a polypeptide, or as a nucleic acid encoding a transposase that is part of a plasmid. The transposase may include a promoter, regulator, or open reading frame.

Embodiments include a cell comprising: a transposase or a nucleic acid encoding a transposase, with the transposase having a sequence with at least 80% identity to SEQ ID NO:29; and a transposon that comprises a target nucleic acid that is mobilizable by the transposase into a genome of a vertebrate cell. The cell may include the target nucleic acid mobilized into the genome of the cell.

Embodiments include a method of introducing a target nucleic acid into DNA in a cell comprising: introducing into the cell (a) a polypeptide that has an amino acid sequence that has at least 80% identity to SEQ ID NO: 29, with the polypeptide possessing binding to a nucleic acid fragment that comprises an inverted terminal repeat sequence of at least one of SEQ ID NO:34 and SEQ ID NO:35, with the polypeptide catalyzing the integration of a target nucleic acid into a vertebrate cell, or (b) a transposase or a nucleic acid encoding a transposase, with the transposase having a sequence with at least 80% identity to SEQ ID NO:29; and a transposon that comprises a target nucleic acid that is mobilizable by the transposase into a genome of a vertebrate cell, or the polypeptide of (a) and the transposase or nucleic acid of (b).

Embodiments include a nucleic acid transposase comprising the 5′ UTR of the transposase located 3′ of the left inverted terminal repeat, as already described.

Embodiments include a target nucleic acid and a selectable marker can be provided on separate transposons and administered to either embryos or cells or animals or humans in unequal amount, where the amount of transposon containing the selectable marker far exceeds (5-10 fold excess) the transposon containing the nucleic acid of interest. Transposases may be administered as described herein. Transgenic cells or animals expressing the nucleic acid of interest can be isolated based on presence and expression of the selectable marker. The nucleic acid of interest and the selectable marker are not genetically linked and may be separated by genetic segregation through standard breeding. Thus, transgenic animals can be produced that are not constrained to retain selectable markers.

Embodiments include a vector that comprises a transposase or a transposon or both a transposase and a transposon. Embodiments include a cell or an animal (including a human animal) that comprise the vector. A plurality of vectors that each comprise one or more target nucleic acids may be provided and may be administered to a cell, an animal, or human patient.

TABLE 1

| (L) Integration Site (R) | | | Chrm Pos | Gene ID | V-Step |
|---|---|---|---|---|---|
| ATGATGCAGCTGGATCCGAT | TA | ATCGGTACCATTTAAATCTG | — | VECTOR | |
| CTACCCAGACTCATTTGATT | TA | actgggaaagtctcttggta | 2 p21 | Intergenic | 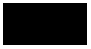 |
| TTTCAATTCTTTTGAATGTA | TA | cctacgaatagaattgctgg | 2 q24 | PLA2R1 | 4+ |
| gttgggaacttaacttgaac | TA | GTATAGAAAGGATGTCCGAA | 2 q37 | Intergenic | 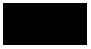 |
| GTCCAGAAGTGAGTTCAGAT | TA | gatcaattctgttagcacct | 3 q25 | Intergenic | 2.5 |
| GTTTTTATTTATCTTGAGTA | TA | taccatgaattggcactgct | 4 q32 | (hmm3072864) | 3.5 |
| gatggttgcattaaacaatt | TA | TGTCCTAAATTATGCACAAT | 5 p13 | Intergenic | 2.5 |
| agacatagatgttacatata | TA | GATTTAGTGTATTGTAGATA | 6 p21 | SUPT3H | 4+ |
| tacatggtagtttaaaatta | TA | CATCACTTTGTATATGGAGC | 6 q14 | Intergenic | 3.5 |
| catctttttatattgttagg | TA | GTAAGTGTATATTTCAAACC | 6 q21 | FYN | 3 |
| GCAGAGGCCTGTGTCAGGTT | TA | aatgtgagctgcaggcagag | 6 q26 | TULP4 | 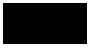 |
| TCAAAGCAAGAAAGATTTAT | TA | gctcgagtctctgcaacaaa | 7 q32 | PODXL | 2.5 |
| gagtggctaagtaggatatt | TA | GGTTCTCAAAGCTAATAGAG | 9 p24 | PCD1LG2 | 2.5 |
| TGTTGTCAAGTTTATTGATA | TA | catcctttaataatgctttt | 9 q22 | FANCC | 3.5 |
| CGCACCAAGTCGATAGTATT | TA | tgctaaagtctctctgaaat | 11 q24 | ETS1 | 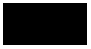 |
| GTACGTATAGATTTGACTGG | TA | tacaaccttcctggggcggc | 12 p11 | PPFIB1 | 3 |
| gatgctagagaatcaacttt | TA | ATTCCAAAACTTGGTACATT | 12 p12 | PLEKHA5 | 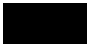 |
| TAATAGTGATGAGTGGTATC | TA | tctccactcaagaaaaatgg | 12 p13 | (hmm15010263) | 4+ |
| gcatccccacagacacacct | TA | CCTGTTCAGTGCAGGCACCT | 12 p13 | Intergenic | 3 |
| CAGCTCTCCCTCTGCCTCCC | TA | ttataagaacactgatgatt | 12 q13 | Intergenic | 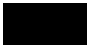 |
| TCTATCATTACCCCATGGCC | TA | gatcatgaaactgagtctta | 12 q24.1 | (hmm1230274) | 3.5 |
| AGAGGAGAGAAGGGAGCTTT | TA | atacagctttcggtcaaaag | 13 q14 | RCBTB1 | 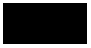 |
| TCCCTATAAGCTCTACCATG | TA | cctacagtcctagggcaga | 14 q22 | Intergenic | 4+ |
| GCAAACCACCATGGCATATG | TA | tagctatgtaacaaacatgc | 15 q11 | Intergenic | 4+ |
| ttggtttgaataactggttt | TA | GTTCAATGTCAACCCTGCAA | 15 q26 | ALDH1A3 | 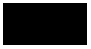 |
| CCCAGAAACCAGCCATAATC | TA | ctcatttagcaaaaatcatg | 17 q21 | NBR1 | 2.5 |
| TAGTTATTTATACTAAGGTG | TA | aatgattgctgtcccactca | 18 p11 | (hmm1912534) | 4+ |
| ttacacatatgatgccatgc | TA | TCTTTATTGTTCTGTAGCTT | 22 q13 | Intergenic | 2.5 |

63% (17/27) in txn units (w/ hypothetical)
48% (13/27) in txn units

What is claimed is:

1. An isolated transposase comprising:
a polypeptide that has an amino acid sequence that has at least 80% identity to SEQ ID NO: 29, with the polypeptide specifically binding to the a nucleic acid fragment that comprises an inverted terminal repeat sequence of at least one of SEQ ID NO:34 and SEQ ID NO:35, with the polypeptide be capable of catalyzing the integration of a target nucleic acid into a vertebrate cell.

2. The transposase of claim 1 wherein the target nucleic acid is integrated into the genome of the vertebrate cell.

3. The transposase of claim 1 comprising SEQ ID NO:29.

4. A nucleic acid encoding the transposase of claim 1.

5. The nucleic acid of claim 4, wherein the nucleic acid is a portion of a plasmid.

6. A cell comprising the transposase of claim 1.

7. A transposon comprising a first nucleic acid fragment with at least 80% identity to SEQ ID NO: 34 and a second nucleic acid fragment with at least 80% identity to SEQ ID NO: 35, wherein the transposon can be specifically bound by a polypeptide having the sequence of SEQ ID NO:29.

8. The transposon of claim 7 further comprising a target nucleic acid fragment that is located between the first nucleic acid fragment and the second nucleic acid fragment, wherein the target nucleic acid is mobilizable by the polypeptide to be integrated into the genome of a vertebrate cell.

9. A cell comprising the transposon of claim 8.

10. A gene transfer system to introduce DNA into the DNA of a cell comprising:
a transposase or a nucleic acid encoding a transposase, with the transposase having a sequence with at least 80% identity to SEQ ID NO:29; and
a transposon that comprises a target nucleic acid that is mobilizable by the transposase into a genome of a vertebrate cell.

11. The system of claim 10 wherein the transposon comprises SEQ ID NO:34 and SEQ ID NO:35.

12. The system of claim 10 wherein the target nucleic acid further comprises a promoter.

13. The system of claim 10 wherein the transposase has the sequence of SEQ ID NO:29.

14. The system of claim 10 wherein the transposase is provided as RNA.

15. The system of claim 10 wherein the transposase is provided as a polypeptide.

16. The system of claim 10 wherein the transposase is provided as the nucleic acid encoding a transposase and is part of a plasmid.

17. A cell comprising:
a transposase or a nucleic acid encoding a transposase, with the transposase having a sequence with at least 80% identity to SEQ ID NO:29; and
a transposon that comprises a target nucleic acid that is mobilizable by the transposase into a genome of a vertebrate cell.

18. The cell of claim 17 further comprising the target nucleic acid mobilized into the genome of the cell.

19. A method of introducing a target nucleic acid into DNA in a cell comprising:
introducing into the cell
(a) a polypeptide that has an amino acid sequence that has at least 80% identity to SEQ ID NO: 29, with the polypeptide possessing binding to a nucleic acid fragment that comprises an inverted terminal repeat sequence of at least one of SEQ ID NO:34 and SEQ ID NO:35, with the polypeptide catalyzing the integration of a target nucleic acid into a vertebrate cell, or
(b) a transposase or a nucleic acid encoding a transposase, with the transposase having a sequence with at least 80% identity to SEQ ID NO:29; and a transposon that comprises a target nucleic acid that is mobilizable by the transposase into a genome of a vertebrate cell, or
the polypeptide of (a) and the transposase or nucleic acid of (b).
